(19) United States
(12) Patent Application Publication
(10) Pub. No.: US 2006/0198907 A1
(43) Pub. Date: Sep. 7, 2006

(54) Pharmaceutical, therapeutic, and dietary compositions derived from Lagerstroemia speciosa L. plant

(76) Inventors: Alex Moffett, Chatsworth, CA (US); Parag Shah, Bangalore (IN)

Correspondence Address: GREENBERG TRAURIG, LLP, 1900 UNIVERSITY AVENUE, FIFTH FLOOR, EAST PALO ALTO, CA, 94303, US

(21) Appl. No.: 11/317,939

(22) Filed: Dec. 22, 2005

Related U.S. Application Data
(60) Provisional application No. 60/638,873, filed on Dec. 22, 2004.

(51) Int. Cl. A61K 36/18

(52) U.S. Cl. 424/725

(57) ABSTRACT

The present invention relates to pharmaceutical, therapeutic, and dietary compositions derived from leaf of the Lagerstroemia speciosa L. plant and the novel extraction processes used to produce these controlled blends of varying composition with respect to corosolic acid, gallotannins, ellagitannins, and valoneic acid dilactone. Embodiments of the invention include an approximately 15.5% to about 98.5% mixture of a corosolic acid rich banaba leaf extract in a tannic acid enriched base for novel combinations for pharmaceutical, therapeutic and/or dietary compositions that yield healthful benefits. Some embodiments of the invention do not include corosolic acid, or include it at a low concentration in combination particularly enriched in other compounds. Such extract compositions that do not contain corosolic acid, are the product of extraction process that yield controllably increased ratios of gallotannins, ellagitannins, and valoneic acid dilactone. Such compositions, both those containing corosolic acid and those lacking corosolic acid are efficacious in effecting control of blood glucose levels, and can be further enhanced in their efficacy by post production and formulation strategies that utilize nanotechnological approaches, targeted deliver enteric coatings, and specialized microenacapsulations. With respect to the individual compounds of these extract compositions, such compounds show combined effects that are both additive and synergistic regarding improved glucose cellular uptake, reduction in blood glucose, insulin efficiency and the simultaneous reduction in assimilation of sugars and starches, and weight loss. Further, additive and synergistic effects are a potential benefit when these compositions are combined with other second therapeutic agents.

### RELATED APPLICATIONS

This patent application claims priority to U.S. Provisional Patent Application No. 60/638,873, of Moffett and Shah, filed on Dec. 22, 2004.

### FIELD OF THE INVENTION

The present invention relates to pharmaceutical, therapeutic, and dietary compositions derived from the leaf of the Lagerstroemia speciosa L. plant and the novel extraction processes used to produce those compositions.

### BACKGROUND OF THE INVENTION

Banaba is the common name of the herb Lagerstroemia speciosa, which belongs to the family Lythraceae, in the order Myrtaceae. Lagerstroemia, “The Banaba Tree” is a tropical, flowering Lagerstroemia tree that is indigenous to India, the Philippines, Australia, China, and Southeast Asia. Its large ornamental leaves are oblong, pointed at the ends, and turn red when they are mature. The flowers are scentless, bright pink to purple, and the stamens appear wrinkled. The banaba fruits are woody and contain seeds that are many winged.

Banaba leaf has traditionally been used as a remedy for the symptoms associated with elevated blood glucose levels. Traditionally, a simple water infusion or tea is brewed from the leaf and flower of the Lagerstroemia speciosa L. plant and the juice obtained is consumed. Such infusions and/or teas have been administered for balancing blood sugar levels and as treatments for diabetes and hyperglycemia.

It is believed that the banaba leaf and flower extract induces glucose transport from the blood into cells of the body. Studies have shown that an extract from Lagerstroemia speciosa L. stimulates glucose transport activity in Ehrlich ascites tumor cells (Murakami C, et al. “Screening of plant constituents for effect on glucose transport activity in Ehrlich ascites tumour cells”, Chem. Pharm. Bull. (Tokyo) December 1993; 41(12): 2129-31). Corosolic acid (2-hydroxyursolic acid) is generally considered to be the active ingredient in Lagerstroemia speciosa L. extracts. Recently, a banaba extract standardized to a 1% corosolic acid concentration was shown to reduce blood glucose levels in subjects suffering from Type II diabetes (Judy, William V., et al.: “Antidiabetic activity of a standardized extract (Glucosol™) from Lagerstroemia speciosa Leaves in Type II diabetics—A dose-dependence study”, Journal of Ethnopharmacology 87 (2003) 115-117). Accordingly, banaba leaf extracts containing corosolic acid are thought to have regulatory effect on levels of sugar and insulin in the blood, and thus may be useful for preventing and/or treating diabetes, hyperglycemia, and obesity. (See Kakuda T, et al., “Hypoglycemic effect of extracts from Lagerstroemia speciosa L. leaves in genetically diabetic KK-AY mice”, Biosci Biotechnol Biochem. February 1996; 60(2): 204-8 and Suzuki Y, et al., “Antiobesity activity of extracts from Lagerstroemia speciosa L. leaves on female KK-Ay mice”, J Nutr Sci Vitaminol (Tokyo). December 1999; 45(6): 791-5.)

It is estimated that over 15 million people in the United States suffer from diabetes mellitus (DM), and it is the seventh leading cause of death in the United States. Diabetes is a complex chronic disorder of carbohydrate, fat, and protein metabolism that results primarily either from a partial or complete lack of insulin secretion by the beta cells of the pancreas, or from defects in cellular insulin receptors. Accordingly, the disorder revolves around an inability to control blood glucose levels, which results in elevated glucose levels, and in turn, may lead to secondary health problems such as: hyperglycemia, arteriosclerosis, diabetic retinopathy (possibly leading to blindness), cataracts, nephropathy, increased risk of infections, hypertension, nerve disease, risk of amputations, impotence, diabetic ketoacidosis, and dementia.

There are two primary types of diabetes mellitus, based on the level of insulin production by the pancreatic beta cells: Type I Diabetes, which represents between 5 and 10% of the total diabetic population, generally emerges in childhood and is known as Juvenile Diabetes (JD) or Insulin Dependent Diabetes Mellitus (IDDM). In IDDM, the medical consensus is that the pancreatic beta cells are damaged by the body's own immune system early in life, and thus little or no insulin is produced. Accordingly, an individual suffering from IDDM is dependent on daily injections of insulin. Type II Diabetes, which represents between 90 and 95% of the total diabetic population, is also known as Non-insulin Dependent Diabetes Mellitus (NIDDM). In NIDDM, the medical consensus is that the pancreatic beta cells do produce insulin, but not in sufficient quantities to maintain healthy blood glucose levels. Associated with insufficient insulin, and compounding the problem, is insulin resistance, a deterioration in the molecular machinery that mediates the effectiveness of insulin function on cells.

The complications of diabetes are fundamentally associated with hyperglycemia, the condition of having too much glucose in the blood. Hyperglycemia can be caused by insufficient insulin, excess consumption of food with simple sugars, insufficient exercise, various illnesses, and excess stress. Hyperglycemia can induce increased aldose reductase activity, which affects sorbitol accumulation, depletes neural myoinositol, and alters Na—K ATPase activity. Hyperglycemia also increases diacylglycerol and protein kinase C activity, which in turn, alters the contractility and hormone responsiveness of vascular smooth muscle, and alters endothelial cell permeability. Moreover, hyperglycemia is associated with accelerated non-enzymatic glycosylation processes that activate endothelial and macrophage receptors for advanced glycosylation endproducts (AGEs), and alters the lipoprotein profile as well as matrix and basement membrane proteins. Ultimately, over time, hyperglycemia and its complications may lead to a coma or death.

Fluctuations in blood sugar and insulin are often related to appetite, hunger, and weight gain. Glucose is the principal energy-supplying nutrient for the body's cells, and thus glucose transport from the blood into cells is of fundamental importance for cell function. On the whole, the balance between glucose intake and storage vs. glucose metabolism and expenditure as energy is a determining factor in the stability of body weight. A long-term positive storage balance leads to weight gain, while a negative balance leads to weight loss. Conversely, the quantity and quality of food intake can affect insulin production and blood sugar levels in the body. Eating foods high in calories and rich in carbohydrates and simple sugars can lead to an increase in both the number and size of adipocytes (fat storage cells). Such increases are associated with insulin resistance, diabetes, hyperglycemia, high cholesterol, high blood pressure and other health risks.

Ordinarily, complex homeostatic mechanisms involving insulin regulate blood sugar levels by controlling glucose uptake and storage into fat and muscle cells, thus keeping the blood glucose level constant. However, these mechanisms can malfunction when over-stressed by internal and/or external environmental stimuli, which, if left unchecked, can result in the conditions of obesity, hyperglycemia, and/or diabetes set forth above. In addition to diabetes, a disease termed “Syndrome X” or “Metabolic Syndrome”, also involves defective glucose metabolism (insulin resistance), elevated blood pressure (hypertension), and a blood lipid imbalance (dyslipidemia). (See Reaven, 1993, Annu. Rev. Med. 44: 121-131.)

There are a variety of different treatments aimed at more effectively regulating insulin secretion and glucose uptake, for those who suffer from abnormal blood glucose levels, including: restrictive diets, oral hypoglycemic drugs, and/or insulin replacement therapy by injection. However, these current treatments can result in adverse effects; for instance, restrictive diets are hard to maintain and can lead to binge eating; oral hypoglycemic drugs can cause head aches, rapid mood swings, and weight gain; and the administration of insulin often lowers blood glucose by promoting the non-selective uptake of glucose by many cells, including adipocytes, which contributes to undesirable weight gain. Hence, continuous treatment modifications and monitoring are often necessary to balance the positive and negative effects of the various different treatment modalities.

The most successful treatments, however, are those that approach this problem from a multiple ways that include restrictive diets, therapeutic drug administration, and food supplementation. Accordingly, since extracts from banaba leaf and flower function much like insulin in helping to control glucose uptake, banaba is thought to have potential an effective supplement in a combinatorial treatment for regulating blood sugar levels, suppressing appetite, and/or enhancing other various treatment regimes for hyperglycemia and diabetes.

Banaba leaf extracts contain a triterpenoid compound, corosolic acid (2-hydroxyursolic acid), that is believed by some to be the active ingredient of the extract. The applicants understand this as a currently accepted view, but believe that it is one that could be modified as information develops that implicates other Lagerstroemia-sourced compounds as having pharmacological activity. U.S. Pat. Nos. 6,485,760 and 6,716,469 to Futoshi Matsuyama disclose methods and compositions for inhibiting an increase in, or lowering, blood sugar levels in a human patient by administering a composition that includes a Lagerstroemia speciosa L. extract with a corosolic acid content of 0.01 to 15 mg per 100 mg (0.01% to 15%) of the concentrate. U.S. Pat. No. 6,784,206 to Udell et al. discloses a method of manufacturing a soft gel capsule that includes a 1% corosolic acid content. Although various banaba extract compositions containing corosolic acid, such as those disclosed in these patents have been proposed as mimics of insulin, these compositions have not completely fulfilled medicinal expectations, given the effectiveness of traditional Banaba leaf teas and infusions. Further, some studies using extracts standardized to contain 1% corosolic acid have failed to show hypoglycemic effects, for example see Hong H and Maeng W J, “Effects of malted barley extract and banaba extract on blood glucose levels in genetically diabetic mice”, J Med Food. 2004; 7(4): 487-490; Edens N and Skelding M B, “Oral administration of Lagerstroemia speciosa extract may delay starch digestion, but does not improve oral glucose tolerance in Zucker diabetic fatty rats”, Diabetes. 2003; 52(suppl. 1): A385-A386) as did another study using a simple hot water extract of the leaves (Suzuki Y et al., “Antiobesity activity of extracts from Lagerstroemia speciosa L. leaves on female KK-Ay mice”, J Nutr Sci Vitaminol. 1999; 45(6):791-795. The reasons for these unfulfilled expectations for extracts of Banaba leaf and discrepant results has not been clear. One possible explanation could involve an incomplete understanding of the active agents within the Banaba leaf, as well as the state of extraction and processing methods available in the industry, which generally focus on corosolic acid as the single active ingredient to the virtual exclusion of other compounds within the banaba leaf extract.

### SUMMARY

The present invention relates to pharmaceutical, therapeutic, and dietary supplement compositions derived from the leaf of the Lagerstroemia speciosa L. (banaba) plant. A first object of the present invention is to provide novel pharmaceutical, therapeutic, and dietary compositions that comprise controlled blends of varying composition with respect to corosolic acid, gallotannins, ellagitannins, and valoneic acid dilactone. Gallotannin is also known by the formal chemical name, penta-O-Galloyl-D-Glucopyranose (PGG), and it exists in both an alpha and a beta form. Some compositions include higher concentrations of corosolic acid, that are balanced with regard to their concentrations of gallotannins, ellagitannins, valoneic acid dilactone, other embodiments include compositions that have low corosolic acid concnetrations, or are absent corosolic acid, or are essentially free of corosolic acid, but enriched in the other aforementioned phytochemicals. “Essentially free” in this sense refers to the absence of corosolic acid by conventional analytical methods, or to concentrations that are very low relative to the aggregate concentration of other phytochemicals, such as about 2% or less than such aggregate concentration.

Accordingly, embodiments of the present invention include novel compositions comprising about a 15.5% to about a 98.5% corosolic acid mixture derived from a novel extraction process of the banaba leaf, compositions that yield surprising health benefits with respect to effectively regulating insulin secretion and glucose uptake. In some embodiments, the novel compositions of the invention include various combinations of mixtures of corosolic acid, gallotannins, ellagitannins, and/or valoneic acid dilactone; wherein the corosolic acid concentration is between about 15.5% to about 80% of the mixture, the combined gallotannin and ellagitannin concentration is between about 20% to about 85% of the mixture, and/or the valoneic acid dilactone concentration is between about 0.01% to about 20% of the mixture. Still other embodiments include compositions in which corosolic acid is absent, or present at a very low concentration, and instead, the active compounds present are tannin-enriched, with the combined species of gallotannins and ellagitannins comprising about 10% to about 98% of the composition. Such embodiments may also further include valoneic acid dilactone.

Included within the first object of providing therapeutic and dietary compositions is the providing of appropriate formulations, with excipients and coatings that support optimal and appropriate delivery of the active agents of the compositions. Tannins are known, for example, to be highly reactive with proteins such as they would encounter in the gut. Accordingly, for example, in some embodiments, enteric coatings are provided, as well as nanotechnological approaches to controlling particle size, to the range of 20 microns, for example, and controlling surface features of the particles. In still other embodiments, microencapsulated coatings are provided that target delivery to regions of the small intestine, for efficient and appropriate delivery.

A second object of the present invention is to provide a method or process for preparing pharmaceutical, therapeutic, or dietary compositions derived from the Lagerstroemia speciosa L. plant that are rich in natural corosolic acids as well as gallotannins, ellagitannins and hydrolysable forms of both primary tannins which yield valoneic acids, and thus yield the holistic benefits of the banaba leaf extract, either alone or with other complementary and enhancing constituents. A third object of the present invention is to provide processes for the preparation of extracts that are free, or substantially free of corosolic acid. These embodiments thus allow for a focused emergence of the therapeutic benefits to be derived from Banaba-derived tannins, including various species of ellagitannins and gallotannins.

Accordingly, in one aspect of the present invention, an economical process for manufacturing pharmaceutical, therapeutic, and/or dietary compositions derived from the leaf of the Lagerstroemia speciosa L. plant is presented. In one embodiment, an economical process is provided that results in a corosolic acid rich extract product that is about 15.5% to about 98.5%, particularly between about 20% to about 80% corosolic acid. In another embodiment, an economical process is provided that results in a tannic acid rich concentration of both gallotannins and ellagitannin-rich extract product that is about 2.5% to about 85%, particularly between about 20% to about 60%, and most particularly about 40% gallotannins and ellagitannins. In still another embodiment, the economic process for extracting enriched corosolic acid is combined with the economic process for extracting enriched gallotannins and ellagitannins to produce a combination product comprising both corosolic acid and highly enriched tanninc acid concentrations with gallotannins present in customizable ratios to naturally occurring ellagitannins. In still other embodiments, methods are provided that yield compositions that are free of corosolic acid, or have it present at very low concentrations. These processes enrich the extract particularly in the concentrations of gallotannins and ellagitannin species. Additionally, any of the processes above can be combined with a process for extracting valoneic acid dilactone so as to produce a combination product comprising a mixture of corosolic acid, gallotannins and ellagitannins that includes specially extracted hydrolysable forms of tannins to increase the nutritional delivery of valoneic acid dilactone. Still other embodiments include compositions in which corosolic acid is absent, or present at a very low concentration, and instead, the active compounds present are tannin-enriched, with the combined species of gallotannins and ellagitannins comprising about 10% to about 98% of the composition. Such embodiments may also further include valoneic acid dilactone and uric acid.

A fourth object of the present invention is to provide methods of treating or providing prophylactic measures for diseases, particularly those that manifest as disorders of glucose metabolism or for promoting a broadly salutary effect on health, even absent overt disease, and a sense of well being due to greater efficiencies in glucose metabolism, maintenance of levels of insulin secretion and blood levels within moderate ranges, appropriate metabolism of sugars and amino acids, and maintenance of an appropriate body weight. Disorders of uric acid metabolism may also be associated with or contributory to diabetes; as such the inventive Banaba-derived compositions may also be appropriate for treatment of such disorders, as may manifest, for example, by high levels of uric acid in the blood. Formulations for all standard routes of administration are provided, including, for example, oral routes, with solid and liquid formulations, and other routes, such as intradermal, intramuscular, intraperitoneal, subcutaneous, epidural, sublingual, intracerebral, intravaginal, transdermal administration. Further with regard to oral administration, inasmuch as the environment of the gut can be critical to the bioavailability of tannins and other compounds, embodiments of treatment include scheduling of administration of the formulations. Thus, for example, a patient, a subject, or direct consumer is advised by treatment methods provided, to self-administer oral formulations between meals, when protein content in the stomach is low.

A fifth object of the invention is to provide therapeutic compositions that combine the extract of Lagerstroemia speciosa with other second agents which have therapeutic or healthful benefits with regard to controlling blood glucose, thereby enhancing the efficiency and benefit that each agent would provide alone. High blood glucose levels, either transiently or chronically, are associated with diabetes mellitus and metabolic syndrome. Diabetes is further associated with hyperuricemia, as such, the inventive Banaba-based extracts may also be combined with second agents for the treatment of hyperuricemia. Second agents, in this usage, infers that the “first agent” is a composition derived from the Banaba plant, according to embodiments of this invention, and that the “second agent” is a compound, or group of compounds, other than those derived from Banaba, such second agents being derived either from other natural sources or by way of synthesis, or by way of conventional pharmaceutical or other health-related production processes. One example is the use of compositions comprising hydrolysable tannins at high concentrations. Another is hibiscus extract standardized to ±hydroxycitric acid (HCA), from various natural sources such as Hibiscus, Green Tea, or Garcinia mangostana. Further, the inventive compositions described herein could be used in combination with any of the standard pharmaceutical agents used to control blood glucose levels, including insulin itself, or other oral antidiabetic agents.

Embodiments of the present invention are based on the concept of using extraction technologies that are targeted for specific groups and subgroups of phytochemical compounds within a single natural source, such as the Banaba leaf, that possess different polarities. Such targeted strategies produce a precursor extracts or separate extract fractions that serve as components in the assembly of a finished novel extract composition that can be produced only by way of (1) separating extraction processes and then (2) reblending the resulting extracts into a finished extract composition. Such modularized subprocesses and blending yields products that cannot be delivered by conventional single-path extraction concepts or technologies. By customizing the extraction media to target either polar or nonpolar constituents within a single natural source, or possibly another combitorial ratio of solvent polarities of the extraction medium to capture a “blend” of phytochemicals designed for further blending with other “targeted construction extracts”, final extract compositions with compound-by-compound control over phytochemical concentrations emerge that are novel and efficacious as medicinal agents.

The foregoing and other objects, advantages, and characterizing features will become apparent from the following description of certain illustrative embodiments of the invention. While the formulations of the present invention have proven to be particularly useful in the area of pharmaceutical, therapeutic, and dietary compositions, those skilled in the art can appreciate that other formulations and mixtures can be used in a variety of different applications and in a variety of different areas of manufacture to satisfy a wide-ranging variety of pharmaceutical and medicinal needs.

### BRIEF DESCRIPTION OF THE FIGURES

FIG. 1 is a flow chart for the alcohol extraction of the Banaba leaf.

FIG. 2 is a flow chart for Product #1 in the Banaba extraction process.

FIG. 3 is a flow chart for Product #2 in the Banaba extraction process.

FIG. 4 is a flow chart for Product #2A in the Banaba extraction process.

FIG. 5 is a flow chart for Product #2B in the Banaba extraction process.

FIG. 6 is a flow chart for Product #3 in the Banaba extraction process.

FIG. 7 is a flow chart for Tannin enrichment process.

FIG. 8 is a flow chart for Valoneic Acid extraction process.

FIG. 9 depicts the formula for Banaba extract product Lagerstannin.

FIG. 10 depicts the formula for Banaba extract product Lagerstannin B.

FIG. 11 depicts the formula for Banaba extract product Lagerstannin C.

FIG. 12 depicts the formula for Banaba extract product Lagerstroemin.

FIG. 13 depicts the formula for Banaba extract product Flosin A.

FIG. 14 depicts the formula for Banaba extract product Flosin B.

FIG. 15 depicts the formula for Banaba extract product Reginin A.

FIG. 16 depicts the formula for Banaba extract product Reginin B.

FIG. 17 depicts the formula for Banaba extract product Reginin C.

FIG. 18 depicts the formula for Banaba extract product Reginin D.

FIG. 19 depicts the time course of blood glucose in groups of rats that were treated with various Banaba extract formulations, following a sucrose loading.

### DETAILED DESCRIPTION OF THE INVENTION

Pharmaceutical, Therapeutic, and/or Dietary Compositions

1. Active Ingredients

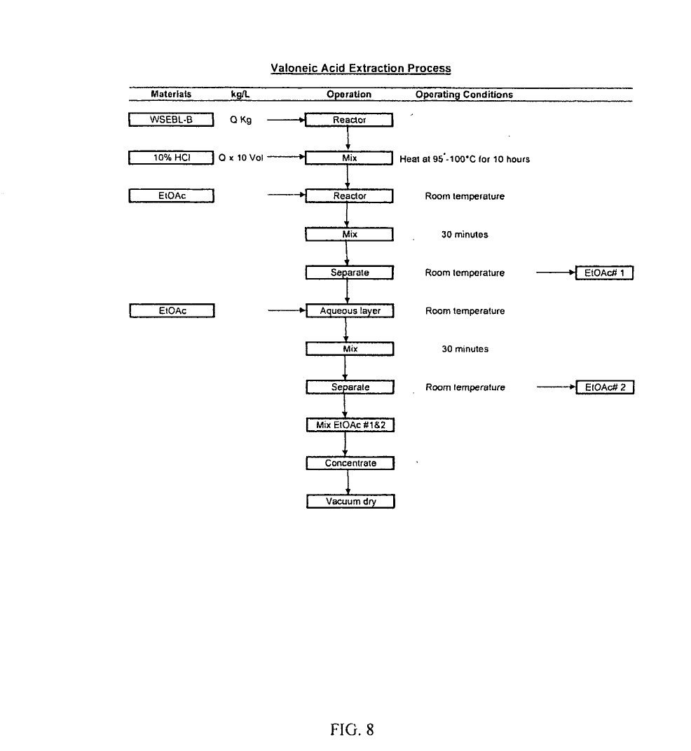

The present invention relates to pharmaceutical, therapeutic, and/or dietary compositions derived from the Lagerstroemia speciosa L. plant (the banaba plant). From its traditional indigenous use, the Banaba leaf is effective in controlling blood glucose levels, and thereby acting as an antidiabetic agent. As described in the background, corosolic acid has drawn attention as a candidate for being the active agent, or an active agent responsible for the antidiabetic action of the leaf and traditional extracts of the leaf. In spite of data supportive of this efficacy of corosolic acid in this regard, the data have not been fully consistent with this theory placing corosolic acid in the center of the Banaba leaf's medicinal properties. The inventors, while believing that corosolic acid may be a significant pharmaceutical agent, have considered the possibility that other phytochemical compounds present in the Banaba leaf may be important contributors to the totality of the potential offered by the Banaba leaf. Their studies, involving field work regarding the phytochemical profile of Banaba leaves at varying states of maturity, and from different locales, and harvested at varying season, as well as studies of various methods of extraction, qualitative and quantitative analysis of extracts by HPLC, as well as animal studies, have drawn their attention to the host of tannins present in the Banaba leaf, as well valoneic acid dilactone. Tannins prominently include ellagitannins and gallotannins, which have been extracted, identified, and quantified (see description below under “examples”, as well as FIGS. 8 through 17.

From their reading of disparate studies in the literature, as well as their own work, the inventors theorize that the glucose regulatory effects of valoneic acid dilactone, in particular, as well as tannins, may be mediated, at least in part, by way of the inhibition of xanthine oxidase, an enzyme whose activity can be responsible for high levels of uric acid in the blood. Various studies in the medical literature have noted an association between hyperuricemia with type 2 diabetes, insulin resistance, and metabolic syndrome. The inventors have observed an inhibition of xanthine oxidase activity by valoneic acid dilactone, and to a lesser extent by ellagotannins. Banaba leaf extracts exposed to hydrochloric acid show an increase in the levels of valoneic acid, and it thus follows that Banaba extract in the acid rich environment of the gut would increase the amount of available valoneic acid. Accordingly, the inventors have developed formulations that both include and exclude corosolic acid, in order to allow the exploitation of the benefits of tannin species and valoneic acid dilactone both alone, and in combination with the effects of corosolic acid.

In particular, the compositions of the invention described herein uniquely provide natural compounds comprising extracts derived from the leaf of the banaba plant. In the present invention, it has been discovered that an approximately 15.5% to about 98.5% corosolic acid concentrate extracted from banaba leaf, when combined with an approximately 2.5% to about 85% ellagitannin rich extract product, and formulated into a pharmaceutical, therapeutic, and/or dietary composition, the combinatorial mixture yields a surprising synergistic effect that renders remarkable health benefits with respect to controlling blood glucose levels. These benefits can further be increased by including within the mixture a valoneic acid dilactone product that can also be extracted from the leaf of the banaba plant via forms of hydrolysable tannins that yield valoneic acid dilactone, or via a preprocessing step for Banaba-sourced tannic acids that result in an extract for that hydrolysis steps possess higher concentrations of valoneic acid dilactone.

Accordingly, in one embodiment, the invention is directed to a composition comprising about 15.5% to about 98.5% corosolic acid. In an another embodiment, the invention is directed to a composition comprising about 2.5% to about 85% ellagitannins. In an additional embodiment, the novel compositions of the invention include various combinations of mixtures of corosolic acid, gallotannins, ellagitannins, and/or valoneic acid dilactone; wherein the corosolic acid concentration is between about 15.5% to about 80% of the mixture, the combined gallotannin/ellagitannin concentration is between about 2.5% to about 85% of the mixture, and/or the compositions may include a valoneic acid dilactone component with a concentration that is between about 0.01% to about 20% of the mixture.

2. Formulations and Routes of Administration

The compositions according to the invention may comprise all pharmaceutically acceptable forms normally utilized according to the route of administration (e.g., oral route, sublingual, injection) required to achieve the therapeutic effect desired. Accordingly, compositions of the present invention may be formulated and employed for oral administration, in unit dosage form (e.g., in hard or soft gelatin encapsulated or tablet form), for administration by absorption through epithelial or mucocutaneous linings, for sublingual administration, or for administration via infusion or bolus injection. Readily flowable forms such as solutions and micro-emulsions may also be employed for instance, for intralesional or epidural injection. The compositions of the invention may also be formulated for administration by any other convenient route, for example, for intranasal or for inhalation administration, and may be administered by itself or together with another biologically active agent. Compositions in accordance with the invention, however, are typically intended for oral or sublingual administration.

For oral and/or sublingual administration, the subject compositions may be formulated into any therapeutically acceptable form normally employed for such an application. In particular, the subject compositions for oral administration may be formulated in the form of tablets, wafer capsules, gelatin capsules, lozenges, aqueous or oily suspensions, granules, powders, emulsions, other capsules, or they may be formulated as drinkable liquids, syrups, suspensions, or elixirs, for example. These compositions may be formulated according to conventional techniques well known in the art.

Optionally the pharmaceutical, therapeutic, and/or dietary compositions of the invention may contain one or more purified or non-purified additives and adjuvants common in such fields, such as sweetening agents such as fructose, aspartame or saccharin; flavonoids or flavoring agents such as peppermint, oil of wintergreen, or cherry; coloring agents; or preserving agents to provide a more therapeutically palatable preparation. Additionally, the compositions may contain antioxidants, solvents, fillers, dyestuffs, and colorants as well as hydrophilic or lipophilic gelling or active agents.

The amounts of these various additives and adjuvants are those used conventionally in the fields under consideration and range, for example, from about 0.01% to about 90% of the total weight of the composition. Exemplary hydrophilic gelling agents which are suitable include carboxyvinyl polymers (carbomer), acrylic copolymers such as acrylate/alkylacrylate copolymers, polyacrylamides, polysaccharides such as hydroxypropylcellulose, natural gums and clays, and, as lipophilic gelling agents, representative thereof are the modified clays such as bentones, fatty acid metal salts such as aluminum stearates and hydrophobic silica, or alternatively ethylcellulose and polyethylene. Exemplary hydrophilic active agents which may be incorporated include proteins or protein hydrolysates, amino acids, polyols, urea, allantoin, sugars and sugar derivatives, water-soluble vitamins, and starch and other plant extracts. And exemplary lipophilic active agents include vitamins, essential fatty acids, ceramides and essential oils.

Moreover, when in tablet or pill form, the compositions may be coated to delay disintegration and absorption in the gastrointestinal tract thereby providing a sustained action over an extended period of time. A time delay material such as glycerol monostearate or glycerol stearate may be used. Hence, the compositions of the invention can be delivered in a controlled release system. For instance, therapeutically acceptable solvents, thickening agents (for added sustained release), or customized coating to reduce protein reactions, or a combination thereof may also be included. Exemplary solvents according to the invention include the lower alcohols, in particular methanol, ethanol, and isopropanol, as well as most other alkyl alcohols such as butyl alcohol and propylene glycol, and the like. Exemplary thickening agents such as polymeric materials may be used. (See: Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23: 61; see also Levy et al., 1985, Science 228: 190; During et al., 1989, Ann. Neurol. 25: 351; Howard et al., 1989, J. Neurosurg. 71: 105.) Other controlled-release systems that can be used in accordance with the invention are discussed in the review by Langer, 1990, Science 249: 1527-1533. Additionally, a variety of other components may be added to the banaba extract component(s) to make up the final net weight composition.

The oral compositions the invention may additionally include standard vehicles such as mannitol, lactose, starch, magnesium stearate, sodium saccharine, cellulose, magnesium carbonate, and the like. Ideally, the vehicles to be included are of therapeutic grade. Additionally, the compositions of the invention may comprise or be comprised in vehicles that can be liquids, such as water and oils, including those of petroleum, animal, vegetable or synthetic origin, such as peanut oil, soybean oil, mineral oil, rice bran oil, sesame oil and the like. Water is a typical vehicle when the compound of the invention is administered intravenously. Saline solutions and aqueous dextrose and glycerol solutions can also be employed as liquid vehicles, particularly for injectable solutions.

The compositions of the invention may also be comprised in vehicles that are gum acacia, gelatin, starch paste, talc, keratin, colloidal silica, bees wax, urea, and the like. Suitable vehicles also include excipients such as starch, glucose, lactose, sucrose, malt, rice, flour, chalk, silica gel, sodium stearate, glycerol monostearate, sodium chloride, dried skim milk, glycerol, propylene, glycol, methanol, ethanol, propanol, and the like. In addition, auxiliary, stabilizing, thickening, lubricating and coloring agents may also be used. The present compositions, if desired, can also contain minor amounts of wetting or emulsifying agents, or pH buffering agents.

Accordingly, the compositions of the invention can take the form of solutions, suspensions, emulsion, tablets, pills, pellets, capsules, gelatin capsules, capsules containing liquids, powders, sustained-release formulations, suppositories, emulsions, aerosols, sprays, suspensions, or any other form suitable for use. In one particular embodiment, the pharmaceutically acceptable vehicle is a capsule (see e.g., U.S. Pat. No. 5,698,155). Other examples of suitable pharmaceutical vehicles are described in “Remington's Pharmaceutical Sciences” by E. W. Martin. If the banaba leaf extract mixture is complemented with juice concentrates, then a liquid beverage is a convenient delivery form. When administered to a subject, the compositions of the invention and therapeutically acceptable vehicles are typically sterile. The amounts of the various constituents of the compositions according to the invention are those conventionally used in the fields under consideration and can be formulated into various compositions according to intent by means and in combinations that are well known in the arts.

The appropriate mode of administration is left to the discretion of the practitioner, and will depend, of course, on the specifics of the patient and the medical condition. In most instances, administration will result in the release of the compositions of the invention into the bloodstream. Typically, administration is via an oral route, however, administration can be systemic or local. This may be achieved, for example, and not by way of limitation, by local infusion, injection or by pulmonary administration by use of an inhaler or nebulizer, and formulation with an aerosolizing agent, or via perfusion in a fluorocarbon or synthetic surfactant.

In certain embodiments of the present invention, the compositions of the invention can be used in combination therapy that include at least one other therapeutic agent. The compound of the invention and the therapeutic agent can act additively or, in some cases it may act synergistically. In a particular embodiment, a composition of the invention is administered concurrently with the administration of another therapeutic agent, which can be part of the same composition as the compound of the invention or a different composition. In another embodiment, a composition of the invention is administered prior or subsequent to administration of another therapeutic agent. Examples of such second agents include gymnemic acids, extracts of Fenugreek and particular phytochemicals isolated therefrom, bitter melon, Heartwood extracts, Salacia extracts, and other phytochemicals, particularly those that benefit glucose regulation, as well as combined amino acid supplementation, any of which may function additively or synergistically in enhancing the over all benefits of this invention.

As many of the disorders for which the compounds of the invention are useful in treating are chronic disorders, in one embodiment combination therapy involves alternating between administering a composition comprising a compound of the invention and a composition comprising another therapeutic agent, e.g., to minimize the toxicity associated with a particular drug. The duration of administration of each drug or therapeutic agent can be, e.g., one month, three months, six months, or a year. In certain embodiments, when a composition of the invention is administered concurrently with another therapeutic agent that potentially produces adverse side effects including but not limited to toxicity, the therapeutic agent can advantageously be administered at a dose that falls below the threshold at which the adverse side is elicited.

The compositions of the invention should be administered in therapeutically effective amounts, typically be in purified form, and may be delivered together with a suitable amount of a therapeutically acceptable vehicle so as to provide the form for proper administration to a subject. The amount of a compound of the invention that will be effective in the treatment of a particular disorder or condition disclosed herein will depend on the nature of the disorder or condition, and can be determined by standard clinical techniques. In addition, in vitro or in vivo assays may optionally be employed to help identify optimal dosage ranges. The precise dose to be employed in the compositions will also depend on the route of administration, and the seriousness of the disease or disorder, and should be decided according to the judgment of the practitioner and each patient's circumstances. However, suitable dosage ranges for oral administration are generally about 0.001 milligram (mg) to about 200 mg of a compound of the invention per kilogram (Kg) body weight.

In particular embodiments of the invention that include corosolic acid, the oral dose is about 0.01 mg-to about 70 mg/Kg body weight, more particularly about 0.1 mg-to about 50 mg/Kg body weight, more particularly about 0.5 mg-to about 25 mg/Kg body weight, and still more particularly, about 1 mg-to about 10 mg/Kg body weight. In a most particular embodiment, the oral dose is 5 mg of a compound of the invention per kilogram body weight. In embodiments of the invention wherein corosolic acid is not present, and tannic acids are the major active agent present, the oral dose is about 10 mg-to about 250 mg/Kg body weight, more particularly, the dose is about 20 mg-to about 225 mg/Kg body weight, and still more particularly about 40 mg-to about 200 mg/Kg body weight. The dosage amounts described herein refer to total amounts administered; that is, if more than one compound of the invention is administered, the preferred dosages correspond to the total amount of the compounds of the invention administered. Oral compositions preferably contain about 10% to about 95% active ingredient by weight. When dosage is normalized to the level of tannins (ellagitannins and gallotannins, combined) or valoneic acid dilactone (as is appropriate when the inventive compositions are those lacking corosolic acid, or having corosolic acid at low concentration) the dosages are higher than those described above for corosolic acids by a factor of several-fold, for example 3-fold, 10-fold, 100-fold, or more.

Recommended dosages for intradermal, intramuscular, intraperitoneal, subcutaneous, epidural, sublingual, intracerebral, intravaginal, transdermal administration or administration by inhalation of coroslic acid-containing embodiments are in the range of 0.001 mg-to 200 mg/Kg body weight. Suitable dosage ranges for intravenous (i.v.) administration are 0.01 mg-to 100 mg/Kg body weight, 0.1 mg-to 35 mg/Kg body weight, and 1 mg-to 10 mg/Kg body weight. Suitable dosage ranges for intranasal administration are generally about 0.01 mg/kg body weight to 1 mg/kg body weight. Suppositories, for intrarectal administration, generally contain 0.01 mg to 50 mg of a compound of the invention per kilogram body weight and comprise active ingredient in the range of 0.5% to 10% by weight. Suitable doses of the compounds of the invention for topical administration are in the range of 0.001 mg to 1 mg, depending on the area to which the compound is administered. Effective doses may be extrapolated from dose-response curves derived from in vitro or animal model test systems, as are well known in the art. As described above in the context of oral dosages, the dosages of embodiments of the present Banaba-derived extracts that do not include corosolic acid, but include tannins as their major active ingredient, the dosage range is higher by a factor of 10 to 100.

Accordingly, the compositions of the invention are typically assayed in vitro and in vivo, for the desired therapeutic or prophylactic activity, prior to use in humans. For example, in vitro assays can be used to determine whether administration of a specific compound of the invention or a combination of compounds of the invention is preferred for lowering fatty acid synthesis. The compounds of the invention may also be demonstrated to be effective and safe in in vivo studies, using laboratory animal model systems.

In an additional aspect the invention provides methods of prophylaxis and treatment by administration to a subject or patient a therapeutically effective amount of a composition of the invention. The subject may be a human, as well as non-human animals, typically mammals, such as, merely by way of example, a cow or steer, a horse, a sheep, a pig, a chicken, a turkey, a quail, a cat, a dog, a mouse, a rat, a rabbit, or a guinea pig.

3. An Overview

One embodiment of the invention is a process for extracting high concentrations of corosolic acid (CRA); this can be termed extract process “A”. Next described are the basic water-only extraction processes for total tannins without any CRA content-extract process “B”. Next described are production methods to target gallotannins specifically—extract process “C”. Later described is a different method for collection and segregation of ellagitannins, extract process “D”. One embodiment is for the combination of the high CRA content and blended Banaba leaf tannin complex termed final extract #1.

Some tannins are hydrolyzable in the gut and have valoneaic acid dilactone: an □-amylase—and glucodase inhibiting substructure of the tannins. This biochemically-modified tannin group possesses a mechanism of action that is complementary for weight-management by reducing the assimilation of dietary sugars and starches.

Embodiments of the invention provide improved testing methods for gallotannin and ellagitannin compositions. Disclosed herein are the extent to which each form and the percentages of each tannin group is hydrolyzable. The inventors have observed that the traditional use of Banaba came from water extracts and that the recent discoveries of the mechanisms of actions for gallotannins and ellagitannins-validate this tradition and elucidate a few of the phytochemicals with specific physiological activities. The inventors disclose additive and synergistic benefits that are delivered by both classes of tannins and by the release of valoneaic acid dilactone post hydrolysis.

Some of the methods of practicing the invention focus on selective banaba leaf collection on two occasions during the leaf maturation cycle of this deciduous tree. One peak occurs at the peak of the ellagitannin content and the second at the peak of the gallotannin content. Then, two distinct extractions are utilized for two targeted forms of tannins. Finally, process methods provide for the blending the two extracts together in such a ratio as to significantly increase the percentage of gallotannins in comparison to the natural ratios to the ellagitannins.

Tannins are highly reactive with proteins, and such reactions in the gut between protein and Banaba tannins, post administration, may reduce the bioavailability and subsequent benefits of the tannin content of the extracts, or products made with the extracts. Therefore, disclosed methods of treatment include a schedule of administration for the products relative to total daily dietary intake and daily activity. Embodiments involving treatment methods direct consumers to take the product (tablet, beverage, beverage pre-mix, food, or food supplement) between meals such protein reaction are less likely to occur. Other embodiments involve the inventive Banaba extract being delivered in an enteric capsule, or micro-encapsulated coating to target its delivery to the small intestines for release of the coating use to avoid or significantly limit the reactivity of the tannins with protein. Although these approaches would theoretically reduce the hydrolysis of tannins and target more of the actions of the extract to glucose transport-stimulating (in vitro); pre-adipocyte cell-differentiating (in vitro); PPAR (peroxisome proliferator-activated receptor-gamma) expression-modulating of some of the tannic acids. It is possible to use both forms of extracts in a single product for both mechanisms of action by combining coated and non coated extracts, and specifically a coated extract that had a higher ratio of gallotannins.

One embodiment of the invention includes a composition comprising tannins that are hydrolysable and will convert into a higher percentage of valoneic acid dilactone for the alpha-amylase and glucosidase-inhibiting effects. Because the molecular weight of the tannins affects their bioavailability a post extract production step is included that improves the overall efficacy of the tannic acid-based formula would be to subject the Banaba tannin extracts to a nanotechnological methods to reduce the extracts contents size to less that 20 microns. The additional enteric or microencapsulated coatings further increases total bioavailability by reducing reactions. And finally both technologies can be further improved by regulation of the schedule of administrations.

Methods of Using the Pharmaceutical, Therapeutic, and/or Dietary Compositions of the Invention

In accordance with the invention, a composition of the invention, which may include a therapeutically acceptable vehicle, is administered to a subject, typically a human being, in need thereof; such need may include a subject suffering from a disorder of glucose metabolism, diabetes, hyperglycemia, obesity, pancreatitis, hypertension, Syndrome X, or inflammation. “Treatment” or “treating” refers to an amelioration of a disease or disorder, or at least one symptom thereof, which may or may not be discernible by the subject. “Treatment” or “treating” may also refer to delaying the onset-, or inhibiting the progression of a disease or disorder, either physically, e.g., stabilization of a discernible symptom, physiologically, e.g., stabilization of a physical parameter, or both.

In certain embodiments, the compositions of the invention are administered to a subject, typically a human being, as a preventative measure against such diseases. As used herein, “prevention” or “preventing” refers to a reduction of the risk of acquiring a given disease or disorder. In various embodiments, the compositions of the present invention are administered as a preventative measure to a subject, typically a human being, having a genetic or non-genetic predisposition to a disorder of glucose metabolism, such as: diabetes, hyperglycemia, obesity, pancreatitis, hypertension, Syndrome X, or inflammation.

More specifically, the present invention provides methods for the treatment or prevention of a glucose metabolism disorder, such as: diabetes, hyperglycemia, obesity, pancreatitis, hypertension, Syndrome X, or inflammation comprising administering to a subject a therapeutically effective amount of a composition(s) of the invention that may include a therapeutically acceptable vehicle. As used herein, the term “glucose metabolism disorders” refers to disorders that lead to or are manifested by aberrant glucose storage and/or utilization. To the extent that indicia of glucose metabolism (i.e., blood insulin, blood glucose) are too high, the compositions of the invention are administered to a patient to restore normal levels. Conversely, to the extent that indicia of glucose metabolism are too low, the compositions of the invention are administered to a patient to restore normal levels. Normal indicia of glucose metabolism are well known to those of skill in the art. Treatment need not be restricted to subjects that have been medically diagnosed with one of these particular diseases or conditions. Many people have conditions that are borderline, or may exercise their own prerogative to avail themselves of agents that have demonstrated healthful benefits with regard to maintaining glucose levels, amino acid levels, and metabolic hormone levels in moderate ranges, with minimal variation in peaks and troughs.

Glucose metabolism disorders that the compositions of the present invention are useful for preventing or treating include impaired glucose tolerance; insulin resistance; insulin resistance related breast, colon or prostate cancer; diabetes, including non-insulin dependent diabetes mellitus (NIDDM), insulin dependent diabetes mellitus (IDDM), gestational diabetes mellitus (GDM), and maturity onset diabetes of the young (MODY); Syndrome X; hyperglycemia; hypoglycemia; pancreatitis; hypertension; polycystic ovarian disease; and high levels of blood insulin and/or glucose. The present invention further provides methods for altering glucose metabolism in a subject, for example to increase insulin sensitivity and/or oxygen consumption of a subject. As used herein, “treatment or prevention of Syndrome X or Metabolic Syndrome” encompasses treatment or prevention of a symptom thereof, including but not limited to impaired glucose tolerance, hypertension and dyslipidemia/dyslipoproteinemia and “Altering glucose metabolism” indicates an observable (measurable) change in at least one aspect of glucose metabolism or related factor, including but not limited to total blood glucose content, blood insulin, the blood insulin to blood glucose ratio, insulin sensitivity, and oxygen consumption. In addition to treating or preventing obesity, the compositions of the invention can be administered to an individual to promote weight reduction of the individual.

Compositional embodiments of the invention can be administered to a non-human animal for a veterinary use for treating or preventing a disease or disorder disclosed herein. Such animals may include domestic and undomesticated animals, either living domestically or under human control, or in a wild state. Examples of domestic animals include pets, laboratory animals, and livestock animals. Typically, such animals are vertebrate animals, although invertebrate animals are included as well. Typically, the vertebrate animals are mammals, but other vertebrate classes are included as well, such as reptiles, amphibians, fish, and birds. In addition to veterinary uses, the compounds of the invention may be used to reduce the fat content of livestock to produce leaner meats. For non-human animal uses, the compounds of the invention can be administered via the animal feed or orally as a drench composition.

Process For the Production of Pharmaceutical, Therapeutic, and/or Dietary Compositions From Banaba Leaf Extract

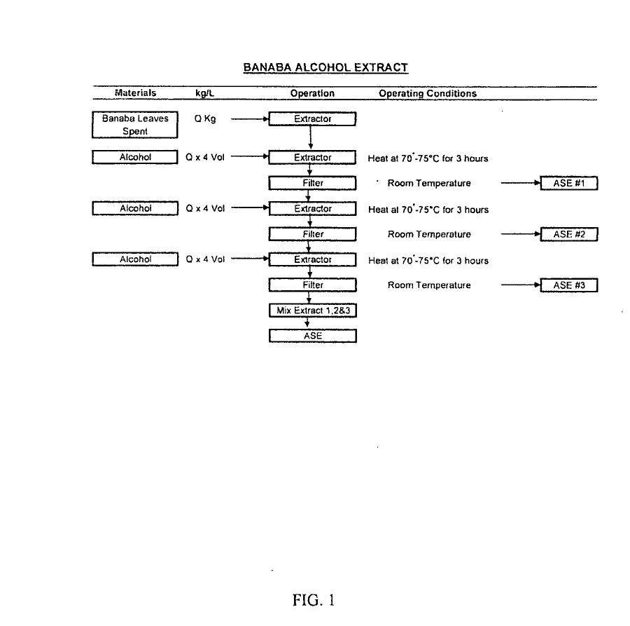

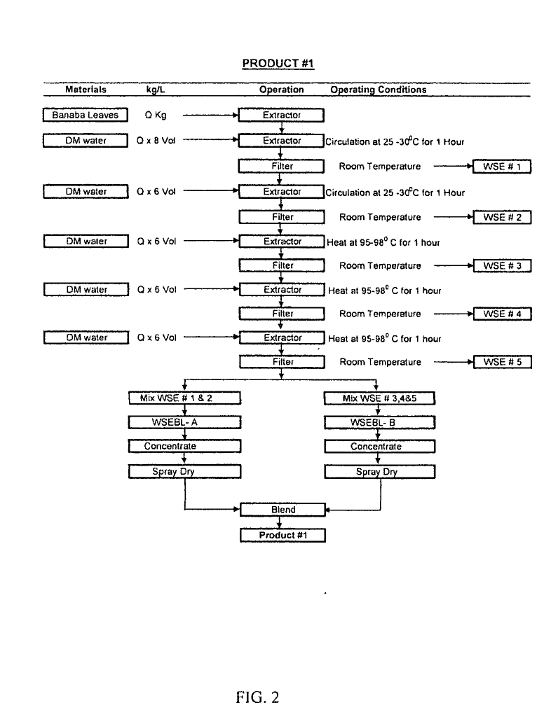

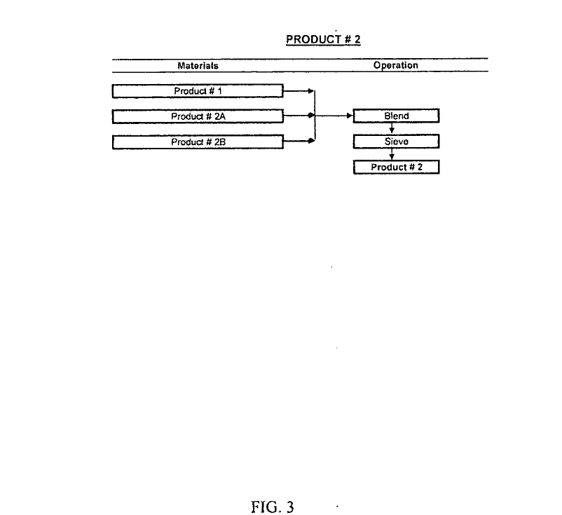

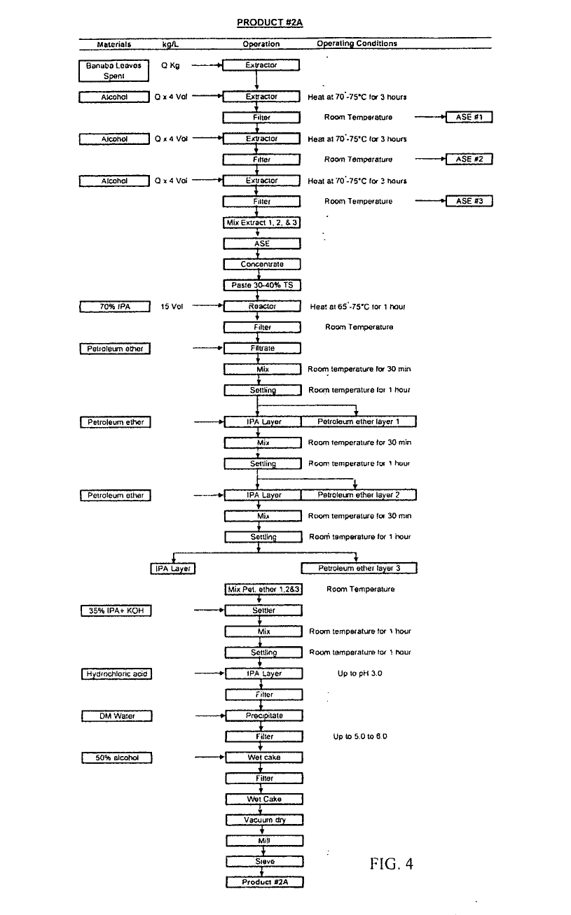

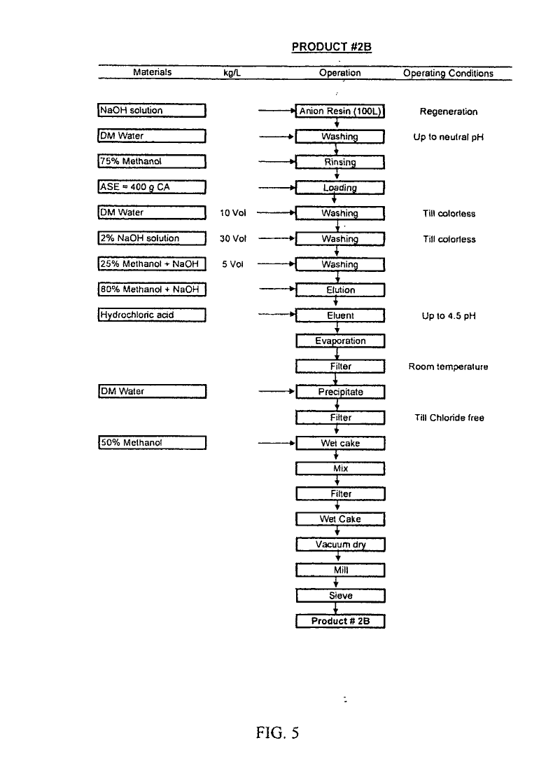

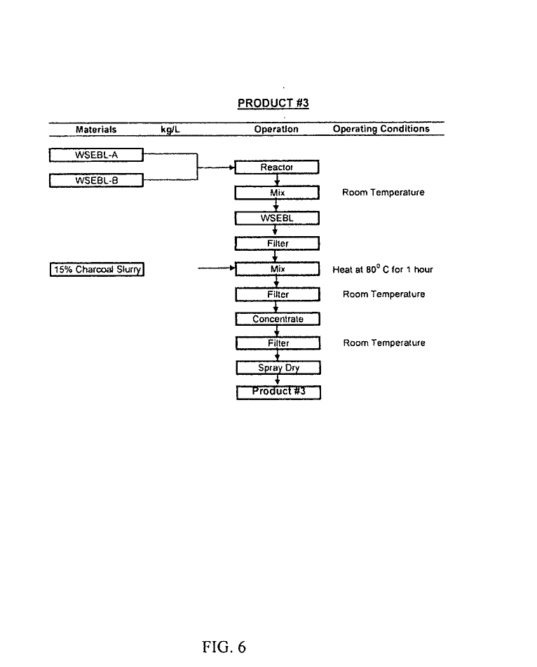

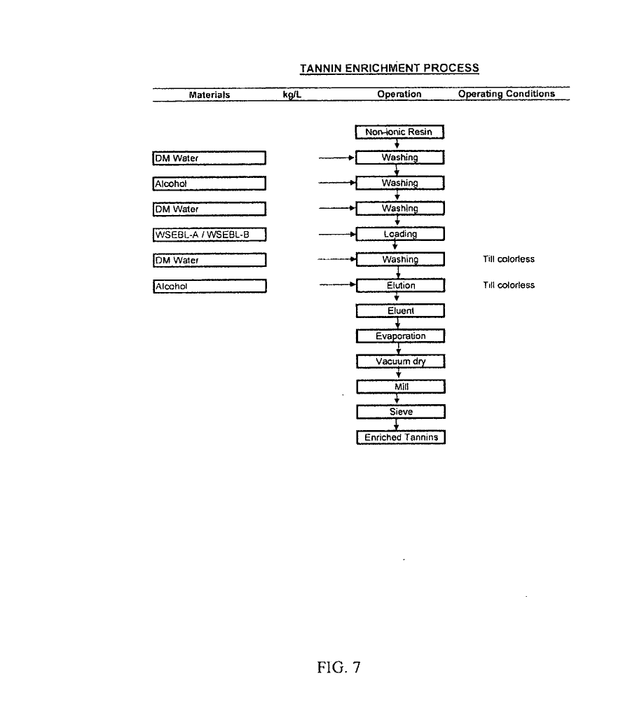

An object of the present invention is to provide processes for preparing pharmaceutical, therapeutic, or dietary compositions derived from the Lagerstroemia speciosa L. plant that are rich in natural corosolic acids, ellagitannins, and/or valoneic acids, such that these compositions yield the holistic benefits of the banaba leaf, either alone or with other complementary and enhancing constituents. Accordingly, in an aspect of the present invention, economical processes for manufacturing pharmaceutical, therapeutic, and/or dietary compositions derived from a leaf extract of the Lagerstroemia speciosa L. plant is provided. The various portions or subprocesses of the totality of the process underlying the extraction of formulations described above, and their analysis and testing, as described below are depicted in FIGS. 1 through 8, each of which is a detailed flow chart the alcohol extraction is shown in FIG. 1, the extraction process for Product #1 is shown in FIG. 2, the extraction process for Product #2 is shown in FIG. 3, the extraction process for Product #2A is shown in FIG. 4, the extraction process for Product #2B is shown in FIG. 5, the extraction process for Product #3 is shown in FIG. 6, the extraction process for the tannin enrichment process is shown in FIG. 7, and the extraction process for the valoneic acid extraction process is shown in FIG. 8.

The processes for preparing the novel compositions of the invention described herein provide novel natural end-products comprising extracts derived from the leaf of the banaba plant using leaves of different maturational levels. In a way, the Banaba extraction process begins in the field, with the collection of the leaves, so as to optimize the composition, particularly of the tannins, in the starting material. The mature green leaf has high ellagitannin content. The inventors have observed that special and significant ellagitannins such as lagerstroemin occur exclusively or predominantly in the mature red Banaba leaf. There are many factors affecting the production and ratios of Banaba tannins such as maturity of the leaf, soil conditions and the rainy season, and whether the leaf is collected directly from the tree, or after it has fallen to the ground.

In the present invention, a specific ratio of Red, Green, and Brown colored leaves are used, and novel extraction methods have been developed that lead to increased yields of beneficial and therapeutic bananba leaf extract products. In one embodiment the methods described herein yield an approximately 15.5% to about 98.5% corosolic acid concentrate extracted from banaba leaf. In another embodiment the methods described herein yield an approximately 88-90% ellagitannins and 10-12% gallotannins of the total tannins present in the product with total tannin content of 2.5% to about 85% gallotannin-ellagitannin rich extract product. Additionally, the novel processing methods described herein can be used to formulate the corosolic acid and/or gallotannin/ellagitannin extraction end-products into a pharmaceutical, therapeutic, and/or dietary combinatorial mixture that yields an additive or synergistic effect which delivers unexpected and significant healthful benefits with respect to controlling blood glucose levels, increasing cellular uptake of glucose, and reduced assimilation of dietary sugars and starches. These benefits can further be increased by including within the mixture a valoneic acid dilactone product that can also be extracted from the leaf of the banaba plant after the hydrolysis of the ellagitannins. Finally, and as described above, the efficacy and healthful benefits of all such inventive Banaba-sourced compositions may be enhanced by co-formulation-, or combined treatment regimens with other so-called second agents, either from natural sources or by way of synthetic processes.

There a number of ellagitannins that have molecular weights between 650 and 1850 daltons. They have between 1 and 4 molecules of ellagic acid (EA) attached to them. An approximated average molecular weight for an ellagitannin species is around 1200 daltons, each compound including an average of 2 molecules of ellagic acid attached. Thus in a hydrolyzed tannin 1% of ellagic acid is equivalent to 2% of ellagitannin. The molecular structures of ellagitanins are attached with their molecular weights. Most of the ellagitannins have valoneaic acid as a part of the molecule. With regard to gallotannins, gallotannin molecular weights are considered at 940 (PGG). There are 5 molecules of gallic acid (GA) attached to. Thus in a hydrolyzed tannin 1% of gallic acid is equivalent to 1.1% of gallotannin.

An understanding of the variation in composition that is associated with variation in the leaf variety is an important feature of being able to control the extraction processes so as to yield optimal quality and precision in the composition of final extraction products. Typical testing data for the different grades of the leaves is shown in the following table. Data such as these represent important criteria for selecting the blend of the different leaves.

| | 10 Hours Hydrolysis | |
|---|---|---|
| Leaf | Leaf | Water Extract |

| Variety | GA (%) | EA (%) | GA (%) | EA (%) |
|---|---|---|---|---|
| Green | 0.29 | 1.69 | 0.72 | 3.32 |
| Red | 0.34 | 1.17 | 0.96 | 2.75 |
| Brown | 0.19 | 1.93 | 1.05 | 4.94 |

An embodiment of the present invention relates specifically to novel processing methods for the extraction of a corosolic acid rich extract product from the leaf of the banaba plant that comprises about a 15.5% to about a 98.5% weight component of a final pharmaceutical, therapeutic, and/or dietary composition. The extraction methods disclosed herein result in the segregation and inclusion of a greater proportion of corosolic acid in the end product of the extract of the leaf. In particular embodiments of the present invention, the extraction process results in a corsolic acid extract product in a concentration amount ranging from between about 20% to about 80%, and most particularly about 40% concentrate of the total weight of a composition mixture of a single pharmaceutical, cosmetic, therapeutic or dermatological composition.

Accordingly, the extraction process according to an embodiment of the invention involves selection of leaves with different maturation levels at ratios in a range of about 10-15% red leaf: about 50-60% green leaf: about 25-35% brown leaf, and more particularly about 10% red leaf: 60% green leaf: 30% brown leaf. The gallic acid content of the various leaves vary with maturity with red leaf having the highest at 20% greater than green leaf and 40% greater than Brown leaf. The red leaf has the lowest ellagitannin content and very low water soluble ellagitannin content. From various experimental works the inventors have concluded that using the above described ratio of leaves yields the optimum quality of extract of the desired range of total tannins and ratio of ellagitannins and gallotannins by way of a three-step process. The first step comprises exhaustively extracting a blend of selected banaba leaf with cool de-mineralized water to extract tannins in such a way as to have an extract with about 50% ellagitannins and 50% gallotannins of the total tannin content. The second step involves the extraction of these spent leaves with water at reflux conditions to extract tannins in such a way as to have an extract with about 95% ellagitannins and 5% gallotannins of the total tannin content. All process steps involving water make use of de-mineralized water, unless noted otherwise. Following water extraction, the third step comprises extracting corosolic acid, by using alcoholic solvents, from the spent leaf after second step. The end result is a banaba leaf extract with a greater concentration of corosolic acid. Both the water and methanol extraction steps according to the invention may include several sub-steps. Although various amounts and volumes are demarcated herein, it should be understood that various other amounts, volumes, and comparable constituents can be substituted and/or added or deleted without departing from the spirit of the invention.

Typically, the aqueous extract is prepared by selection of leaves with different maturation levels at ratios in a range as 10-15% Red leaf: 50-60% Green leaf: 25-35% Brown leaf, and more particularly, 10% Red leaf: 60% Green leaf: 30% Brown leaf. The selected leaf compositions are then subjected to milling and blending of the banaba leaf and then extracting with 25-30° C. water approximately two times. The process for the water extraction comprises charging a known quantity of banaba leaf (e.g., an approximate amount of 750 kg) and an 8× volume (e.g., 6000L) of water into a suitable extractor. Circulate the water for 1 hour and drain to a clean tank as water soluble extract (WSE #1). An additional 6× volume of water (e.g., 4500 L) is then added to the original extractor. Circulate the water for 1 hour and drain to a clean tank as water soluble extract (WSE #2). An additional 6× volume of water (e.g., 4500 L) is then added to the original extractor The mixture is then heated by supplying steam in the outer jacket of the extractor and refluxing for about 1 hour at a reflux temperature of about 95-98° C. under continuous circulation of the water. After heating, the reflux is stopped, the mixture is drained, and the water soluble extract (WSE # 3) is placed into a clean tank. An additional 6× volume of water (e.g., 4500 L) is then added to the original extractor and the resultant mixture is then heated again, under reflux conditions, up to 95-98° C. and left for up to an additional 1 hr, under continuous circulation. After about 1 hour, the extractor is then drained and the extract (WSE # 4) is collected. This step is then repeated at least once more to give extract (WSE # 5). The extracts WSE #s 1 & 2 are then combined and stored in a cleaned container Labeled as “water soluble extract of banaba leaf” WSEBL-A. Similarly the extracts WSE #s 3, 4, and 5 are then combined and stored in a separate cleaned container Labeled as “water soluble extract of banaba leaf” WSEBL-B and they are used to make the gallotannin and ellagitannin extracts respectively. The WSEBL-A & WSEBL-B are concentrated and spry dried individually and then blended in a specific ratio to give Product # 1.

Typically, the water extraction process operates as follows:
1. charging a known quantity of selection of Banaba leaves with different maturation levels at ratios in a specific range in to a suitable extractor,
2. charging approximately 8 volume of water in to the extractor,
3. Circulate the water for 1 hour.
4 draining and filtering the extract to a clean tank as water soluble extract (WSE #1).
5. adding an additional 6× volume of water to the original extractor,
6. circulating the water for 1 hour, drain and filtering the extract to a clean tank as water soluble extract (WSE #2),
7. adding an additional 6× volume of water to the original extractor,
8. heating the extractor by supplying steam in the outer jacket, and refluxing for 1 hr at a temperature (95-98° C.) under continuous circulation of water,
9. filtering the extract (Water Soluble Extract #3) and storing in a suitable, cleaned container,
10. repeating steps # 7 to 9 two more times (Water Soluble Extract #4 & 5),
11. combining the WSE#1 & 2, Water Soluble Extracts (now termed the WSEBL-A) and storing in clean container,
12. combining the three Water Soluble Extracts WSE#3, 4 & 5 (now termed the WSEBL-B) and storing in clean container,
13. concentrating and spray drying the WSEBL-A & WSEBL-B individually,
14. testing the WSEBL-A & WSEBL-B to check the ratio of ellagitannins and gallotannins, and

15. blending the extracts in a specific ratio to give Product # 1 with the following properties:

| | Gallotannin Content | Ellagitannin Content |
|---|---|---|
| Product | (as % of total tannins) | (as % of total tannins) |
| Product # 1 | 10-12% | 88-90% |

Typically, the alcohol extraction is conducted after the water extraction of the spent banaba leaf, with a suitable alcohol solvent in an appropriate concentration, e.g., 100% methanol, about 90% ethanol, or about 90% isopropanol, or the like. Typically, the process for the alcohol extraction comprises charging a 4× volume of alcohol solvent into the extractor with the water spent banaba leaf at a volume that is approximately 1-3 cms above the soaking level of the raw material. Reflux is initiated and heat is started by supplying steam to the outer jacket of the extractor for about 3 hours at approximately 70-75° C. under continuous circulation. After about 3 hours the heating and circulation are stopped and the alcohol extract is filtered from the extractor, to yield alcohol soluble extract # 1. These steps are repeated at least twice more (yielding ASE #s 2 and 3) and all three (or more) Alcohol Soluble Extracts are then combined and stored in a cleaned container.

Typically, the alcohol extraction process operates as follows:
1. charging approximately 4 volume of alcoholic solvent into the extractor, a volume that should be at least about 2 cm above the soaking level of the raw material,
2. heating by supplying steam in the outer jacket of the extractor and refluxing for 3 hr at reflux temperature under continuous circulation,
3. filtering the alcohol extract (Alcohol Soluble Extract #1) from the extractor,
4. repeating step # 1 to 3 twice more (Alcohol Soluble Extracts # 2 and 3 are obtained), and
5. combining all the three Alcohol Soluble Extracts.

The resultant ASE products (from step #5 above) are then enriched by at least one of two additional methods. The first method involves a solvent-solvent extraction process using different solvents, which have different polarities, and/or then by chromatography using an ion exchange resin column. These methods allow for the removal of impurities without removing the corosolic acid from the extract, thus allowing for a more highly concentrated corosolic acid end product.

The solvent-solvent extraction process involves the further processing of the alcohol soluble extract (ASE) end products of the alcohol extraction process set forth above. First, the combined ASE (from step 5 above) is filtered through a 20 micron filter. The ASE is then charged in to a suitable reactor and heated by using steam up to a distillation temperature (95-98°). Distillation is continued until the alcohol is distilled off and the resultant product is obtained, a thick paste of 30-40% total dissolved solids (TDS). This paste is then charged in to a suitable reactor with 15× volume of 70% isopropanol or its equivalent. The reactor is then heated to a reflux temperature (95-98° C.) and refluxed for about 1 hr under constant stirring. After about 1 hr the extract is cooled to room temperature and filtered through a sparkler filter. The resultant isopropanol solution is then charged in to a settler, an equal volume of petroleum ether is added to the settler, and the mixture is stirred for 30 minutes and then allowed to settle for about 1 hr. After settling, the bottom alcohol layer is collected and placed into a suitable container, and the petroleum ether layer is removed from the settler. The isopropanol layer is again charged in to the settler and another equal volume of petroleum ether is added to the settler and the mixing, settling, and collecting steps are repeated at least twice more, after which all three petroleum ether layers are collected in to the settler and the resulting volume is measured. Half the volume of 35% isopropanol containing 0.3% potassium hydroxide is then charged in to the settler, the alkaline isopropanol -petroleum ether mixture is stirred for about 1 hour and then allowed to settle for about an additional 1 hour, to allow for layer separation. The bottom alkaline-isopropanol layer is then collected and charged in to a suitable glass lined reactor. Hydrochloric acid is slowly added to the reactor under constant stirring until the resultant pH is about 3.0. The precipitation formed in reactor is checked, then filtered through a nutch filter and washed with water until the pH is about 5.0 to about 6.0. The precipitate is again suspended in 50% alcohol (for example, methanol, ethanol) and made into a slurry which is filtered through a nutch filter. The resultant wet cake is vacuum dried, milled, and sieved to a desirable particle size. The product (Product 2A) thus obtained is an off-white banaba extract containing about 15.5% to about 98.5% corosolic acid.

Typically, the solvent-solvent extraction process operates as follows:
1. filtering the combined alcohol soluble extracts through a 20 micron filter,
2. charging the alcohol soluble extract in to a reactor,
3. heating the reactor by using steam up to distillation temperature,
4. continuing the distillation until the alcohol is distilled off and resultant product obtained is a thick paste of 30-40% TDS,
5. charging the thick paste in to a suitable reactor,
6. charging 15 volume of 70% isopropanol into the reactor,
7. heating the reactor to reflux temperature and reflux for 1 hr under stirring,
8. cooling the reactor to room temperature and filtering the extract through a sparkler filter,
9. charging the isopropanol solution in to settler,
10. adding equal volume of petroleum ether to the settler,
11. stirring the isopropanol-petroleum ether mixture for 30 minutes,
12. settling the mixture for 1 hr,
13. collecting the bottom alcohol layer into a suitable container,
14. removing the petroleum ether layer from the settler,
15. charging the isopropanol layer into the settler again,
16. repeating the step # 10 to 13 twice more,
17. combining all the three petroleum ether layers into the settler and measuring its volume,
18. charging half volume of 35% isopropanol containing 0.3% potassium hydroxide in to the settler,
19. stirring the alkaline isopropanol -petroleum ether mixture for 1 hour,
20. settling the mixture for 1 hour, for layer separation,
21. collecting the bottom alkaline-isopropanol layer,
22. charging the alkaline-isopropanol layer in to a suitable glass lined reactor,
23. slowly adding hydrochloric acid in to the reactor under constant stirring, in order to reach a pH of about 3.0,
24. checking the precipitation formed in reactor and filtering through a nutch filter,
25. washing the precipitate with water until the pH is about 5.0 to about 6.0,
26. suspending the precipitate again in 50% alcohol (either methanol or ethanol), making it a slurry,
27. filtering the slurry through a nutch filter, and
28. vacuum-drying the resultant wet cake; milling, and sieving to a desirable particle size, creating a product (Product 2A) that is an off-white banaba extract, about 15.5% to about 98.5% corosolic acid.

Typically, the Ion exchange extraction process operates as follows:
1. regenerating the weak anion resin using sodium hydroxide solution (For example, 100 liters of an appropriate resin (e.g., Indion 850, Exchange India Ltd.), with 8 Kg of sodium hydroxide,
2. washing the resin with water until the pH of the washings is neutral,
3. draining the water and rinsing the resin with 75% aqueous methanol,
4. combining all the three methanol extracts of Banaba leaf and loading a volume equivalent to 400 g corosolic acid on the resin,
5. washing the resin with water until colorless(typically, about 10 volumes),
6. washing the resin with 2% sodium hydroxide solution until it is colorless(typically, about 30 volume),
7. washing the resin with five volumes of 25% aqueous methanol containing 1% sodium hydroxide,
8. eluting the resin with 80% aqueous methanol containing 0.5% sodium hydroxide until the corosolic acid content in the eluent becomes less than about 0.5% on dry basis,
9. collecting all the eluents, acidifying to a pH of about 4.5 with hydrochloric acid,
10. evaporating the methanol completely,
11. filtering the slurry obtained through a 10 micron filter cloth,
12. washing the residue with water until the washings become free of chlorides,
13. washing (again) the residue with 50% methanol, and
14. drying the residue under vacuum; the product (Product 2B) thus obtained is an off-white colored Banaba extract containing about 15.5% to about 98.5% corosolic acid.

15. blending the product # 2A and/or Product # 2B with Product # 1 in specific proportions to obtain Product # 2, with the following specifications:

| Product | Corosolic Acid | Total Tannins |
|---|---|---|
| Product # 2 | 15.5%-98.5% | 2.5%-85% |

Another embodiment of the present invention relates specifically to novel processing methods for the extraction of an approximately 88-90% ellagitannins and 10 -12% gallotannins of the total tannins present in the product with total tannin content of an gallotannin-ellagitannin rich extract product from the leaf of the banaba plant in a more water soluble form specifically for beverage application that comprises about a 2.5% to about 85% weight component of a final pharmaceutical, therapeutic, and/or dietary composition. Typically, the extraction methods disclosed herein result in the segregation and inclusion of a specific proportion of gallotannins and ellagitannins in the end product of the extract of the leaf. In particular embodiments of the present invention, the extraction process results in a gallotannin-ellagitannin extract product in a concentration amount ranging from between about 10% to about 80%, and most preferably about 20% concentrate of the total weight of a composition mixture of a single pharmaceutical, cosmetic, therapeutic or dermatological composition. An important second aspect of this is a specific extraction technology to increase the content of the gallotannins beyond what would be considered normal composition ratios of gallotannins if separate and distinct extraction technologies and blending strategies were not utilized in the creation of a proprietary extract compositions design to increase the dose and functionality of the each group of phytochemicals in the Banaba leaf.

Accordingly, the extraction process for enriched gallotannin-ellagitannin extractions, according to the invention, includes an additional two-step process that follows from the water extraction process set forth above. The first step involves an initial reprocessing of the WSEBL-A and WSEBL-B end products derived from the water extraction process in steps 11 and 12 of the water extraction process described above. Test the WSEBL-A & WSEBL-B for gallotannin and ellagitannin content and blend the extract solutions in a ratio so as to achieve a product to test as Product # 1 on dry basis and label as “WSEBL”. The initial reprocessing step may include several sub-steps. Initially the WSEBL end product is filtered through a sparkler filter stuffed with hyflow supercel powder, and then through a 20 micron filter to remove undesirable matters from the extract. The WSEBL is then charged into a reactor and steam is passed in through the outer jacket of the reactor to heat the WSEBL up to 80° C. A 15% charcoal slurry is then prepared by mixing 15 kg of activated charcoal and 100 L water. The charcoal slurry is added in to the reactor and the temperature is maintained at about 80° C. for about 1 hr. After 1 hr, the reactor is cooled to room temperature, the WSEBL is filtered through a filter press, and then additionally filtered, sequentially, through both a 5 micron and a 0.5 micron filter, steps that improve the clarity of the WSEBL. The filtrate is then collected in to a clean holding tank and any water is distilled from the WSEBL in a falling film evaporator and concentrated to 20 to 22% TDS. The WSEBL is once again filtered, this time, through a hyflow supercel bed, a step that improves the solubility of the final product. The extract solution is then spray dried. The resultant WSEBL extract, product #3, contains about 10 to about 40% of total tannins.

Typically, the further initial reprocessing of the WSBEL extraction end product process operates as follows:
1. filtering the WSEBL through sparkler filter filled with hyflow supercel powder, and then through a 20 micron filter to remove undesirable matters from the extract,
2. charging the WSEBL into a suitable reactor,
3. passing steam in the outer jacket of the reactor and heating to about 80° C.,
4. preparing 15% Charcoal slurry by mixing 15 kg activated charcoal and 100 L water,
5. adding the charcoal slurry in to the reactor and maintaining the temperature at 80° C. for 1 hr,
6. cooling the reactor to room temperature,
7. filtering, and thereby clarifying, the WSEBL through filter press, and then through a
5 micron filter and then a 0.5 micron filter,
8. collecting the filtrate in a holding tank,
9. distilling off the water from the WSEBL in a falling film evaporator and concentrating to 20 to 22% TDS,

10. filtering, once again, the WSEBL through hyflow supercel bed. This filtration step improves the solubility of the final product. The extract solution is spray dried. The resultant WSEBL extract contains about 10 to about 40% of total tannins This is product # 3 with the following specifications.

| | Gallo tannins content | Ellagi tannin Content | Solubility |
|---|---|---|---|
| PRODUCT | (as % of total tannins) | (as % of total tannins) | in Water |
| Product # 3 | 10-12% | 88-90% | >95% |

The enrichment of total tannins in the WSEBL-A and WSEBL-B from 10% to more than 40% to about 85% is achieved by a non-ionic resin column chromatographic technique. The resin used is selected based on its porosity, pore size and hydrophilic nature. The non-ionic polymeric cross linked adsorptive resins suitable for the use in this invention include, by way of currently available examples, Indion NPA 1;NPA 2; NPA 3 (Ion Exchange India Ltd., Mumbai, India) or Hp 20/21 resins (Mitsubishi Corp. Japan), or Amberlite XAD-4, XAD-16, XAD 1180, XAD 7HP, XAD 761 (Rohm and Haas, USA). The salient desirable features of the resin used include: a moisture holding capacity of 50-55%, a surface area of about 300 to 750 m²/g, a porosity of about 0.8 ml/g, and an effective pore size of 80 to 600 A°. The WSEBL-A contains higher level of gallotannins as compared to WSEBL-B, and is processed using resins with a smaller pore size, such as XAD 4, XAD 16, HP 20 or HP 21, as the Gallotannins have a lower molecular weight. Whereas the WESBL-B is processed using resins with larger pore size (e.g., XAD 1180, XAD HP7, XAD 761 as the ellagitannins have a higher molecular weight.

The chromatographic processing method comprises preparing the WSEBL-A and WSEBL-B according to the method above. The solution of the extract containing 1-5% TDS is suitable for loading the column (the resin/extract ratio is about 10:1 v/w). The resin is suspended in water and packed on to the column. Then the column is washed thoroughly with water and then with any suitable alcoholic solvent, such as methanol, ethanol, or the like. Once again the column is washed with water to remove residual solvent from the surface of the resin. The residual water in the column is removed by gravity evacuation and/or vacuum aspiration. The WSEBL-A or WSEBL-B solution is slowly charged on to the column to maximize adsorption of the desirable tannins on the surface of the resin. The excess solution is drained from the column and collected separately. Unwanted materials from the surface of the resin are cleansed by washing the surface with excess amounts of water until the water washing is colorless. If required, an aqueous buffer solution may also be used for effective cleansing of the resin surface. The desirable tannins are removed from the resin surface by using a selective eluant. For this purpose solvents like methanol, ethanol, and/or other similar alcoholic solvent may be used. The resin is then eluted with the solvent until the eluant is colorless. The total tannins are removed from the eluant by distillation of the eluant solvent in vacuo. The solid obtained after the removal eluant is made in to a powder of desirable particle size. The final product obtained comprises more than 40% total tannins, in particular processes more than 80% total tannins, and in more particular processes, about 85% total tannins.

Typically, the chromatographic process operates as follows:
1. preparing a volume of WSEBL-A or WSEBL-B comprising about 10% to about 40% total tannins. The solution of the extract containing 1-5% TDS is suitable for loading the column. (The resin : extract ratio is 10:1 v/w)
2. suspending the selected resin in water and packing on to the column,
3. washing the column thoroughly with water and then with any alcoholic solvent, such as methanol or ethanol. Washing once again the column with water to remove residual solvent from the surface of the resin,
4. removing the residual water in the column by gravity evacuation and vacuum aspiration,
5. charging the WSEBL-A or WSEBL-B slowly on to the column and thus maximizing adsorption of the desirable tannins on the surface of the resin, and draining and collecting the excess solution from the column separately,
6. cleansing unwanted matter from the surface of the resin by washing the surface with copious amount of water, until the water washing is colorless. If required, aqueous buffer solution may also be used for effective cleansing of the resin surface,
7. removing desirable tannins from the resin surface by using a selective eluant (For this purpose solvent like methanol, ethanol and/or other similar alcoholic solvent is used. Elute the resin with solvent until the eluant is colorless),
8. separating the total tannins from eluant by distillation of the eluant solvent in vacuo, and
9. forming the solid obtained after the removal eluant into a powder of desirable particle size; the final product obtained in the present invention consisting of more than 40% total tannins.
   Valoneaic Acid Process

The spray dried water extract WSEBL-B derived from the water extraction process in step 13 of Para 066 (fix this) is mixed with 10 volume 10% hydrochloric acid in a glass lined reactor. The mixture is heated at 95-1000 C. for 10 hours by supplying steam in the outer jacket of the reactor. Cool down to room temperature after hydrolysis. Transfer equal volume of ethyl acetate into the reactor and mix for 30 minute. Allow settling for 1 hour and separate the ethyl acetate layer. Again transfer equal volume of ethyl acetate into the aqueous layer. Repeat the above process and combine both the ethyl acetate layer in a settler. Wash the ethyl acetate layer with water repeatedly until the pH of the washings become neutral. Concentrate the ethyl acetate layer to 30-40 TDS and dry under vacuum.

Typically, the hydrolysis process operates as follows
1. charging a known quantity of WSEBL-B powder into a glass lined reactor.
2. charging approximately 10 volume of 10% hydrochloric acid into the reactor,
3. heating the extractor by supplying steam in the outer jacket, and refluxing for 10 hours at a temperature (95-100° C.) under continuous circulation,
4. Cooling to room temperature by supplying cooling water in the outer jacket,
5. transferring equal volume of Ethyl acetate into the reactor.
6. mixing for 30 minutes,
7. separating the Ethyl acetate layer,
8. repeating the steps 5-7 and combine all the Ethyl acetate layer, and
9. concentrating the Ethyl acetate layer to 30-40 TDS and dry under vacuum.

These processes can be combined to produce a combined end product that comprises corosolic acid, gallotannins and ellagitannins. Additionally, any of these processes above may be combined with a process for extracting valoneic acid dilactone so as to produce a combination product comprising a mixture of corosolic acid, gallotannins and gallotannins/or ellagitannins that further includes increased quantities of process pre-hydrolyzed valoneic acid dilactone.

Analytical Methods and Examples

The effectiveness in improving general health and wellness of the banaba compositions described herein is demonstrated from the following examples, which are listed for illustrative purposes only and are not intended as limiting instances of prophylactic or therapeutic use. A therapeutic composition of the banaba leaf mixture may be prepared according the embodiments described herein. Hence, in order to further illustrate the present invention and the advantages thereof, the following specific examples are given, it being understood that the same are intended only as illustrative and in not limiting in nature.

1. Analytical Methods, Identification and Quantification of Banaba-Extracted Compounds

The elagitannins and the gallotannins are tested by published testing procedure as detailed in “Chemical Identification of the Sources of Commercial Fructus Chebulae” by Lih-Jeng Juang and Shuenn-Jyi Sheu (Phytochem. Anal. 16, 246-251 (2005). Identification of extracted compounds has been made by HPLC.

Corosolic acid analysis has been performed by HPLC according to the following protocol.

HPLC conditions, reagents, standards, calculations:
Column: Luna C18(2), 5 Micron(4.6×250 mm)
Wave length: 210 nm
Flow rate: 1 ml /minute
Volume of Injection: 20 μl
Temperature: 250 C.±20 C.
System: Isocratic
Run time: 15 min.
0.1% v/v Phosphoric acid: Dilute 1 ml of ortho phosphoric acid to 1000 ml with double distilled water.
Mobile phase: 0.1% v/v Ortho phosphoric acid::Acetonitrile (15:85 v/v)
Solvent: Methanol AR Grade
Standard: Chromadex
Retention time: ˜7.5 min.
Standard preparation: Weigh accurately about 25 mg of in-house reference standard in to a 100 ml volumetric flask, add 50 ml solvent, sonicate for 15 minutes, cool and make up to volume with solvent
Sample preparation: Weigh accurately the required quantity of sample into a 25 ml volumetric flask, add 20 ml solvent, sonicate for 15 minutes, cool and make up to volume with solvent. Ensure that the concentration of active ingredient in sample solution is approximately similar to that of standard solution
Filter both standard and sample solutions through 0.45μ membrane filter and inject.
Note down the area of Corosolic acid from both standard and sample graph.
Calculate as follows: Corosolic acid (%)=(peak area of sample×conc. of standard×purity of std)/(peak area of std.×conc. of sample)

Analysis of Raw Material Samples
Weigh 5 g of powdered raw material into a 250 ml round bottom flask.
Transfer 100 ml of isopropyl alcohol and reflux on a water bath for one hour.
Filter the extract through Whatman filter paper into a 250 ml standard flask.
Repeat the extraction and filtration same way for two more times and make up the volume of extract to 250 ml. Dilute the solution, if necessary, and use as sample solution. Calculate corosolic acid, as above.
   2. Compounds Isolated from Banaba Extract

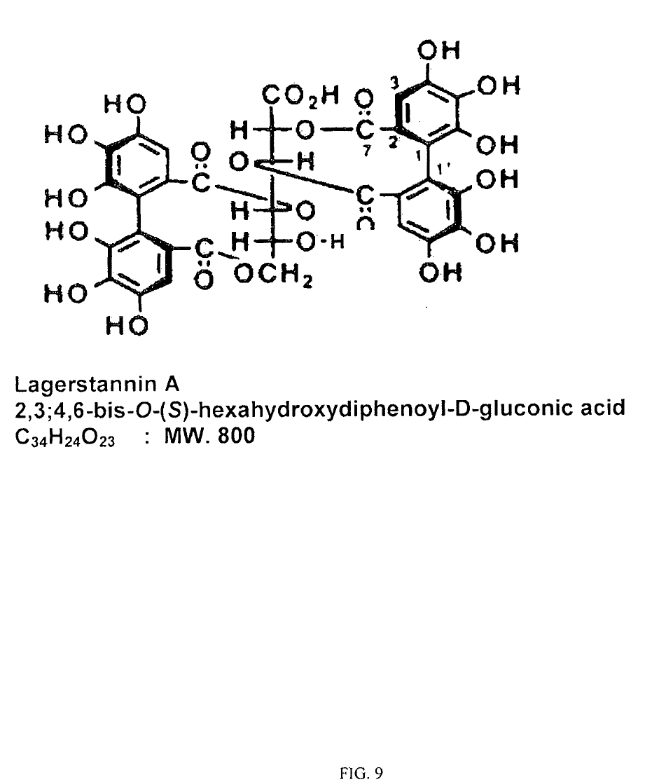

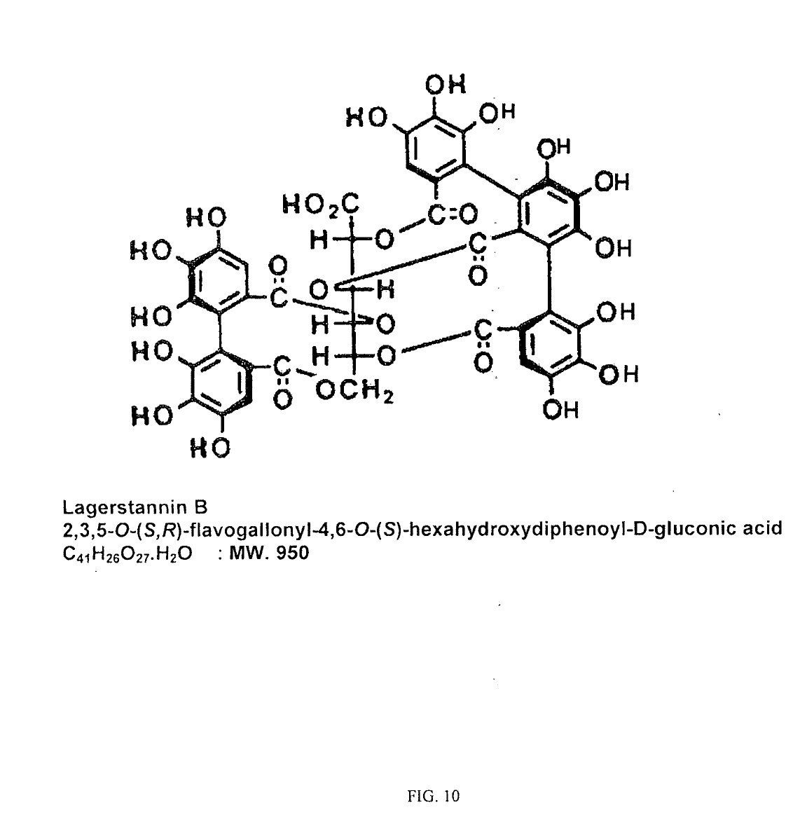

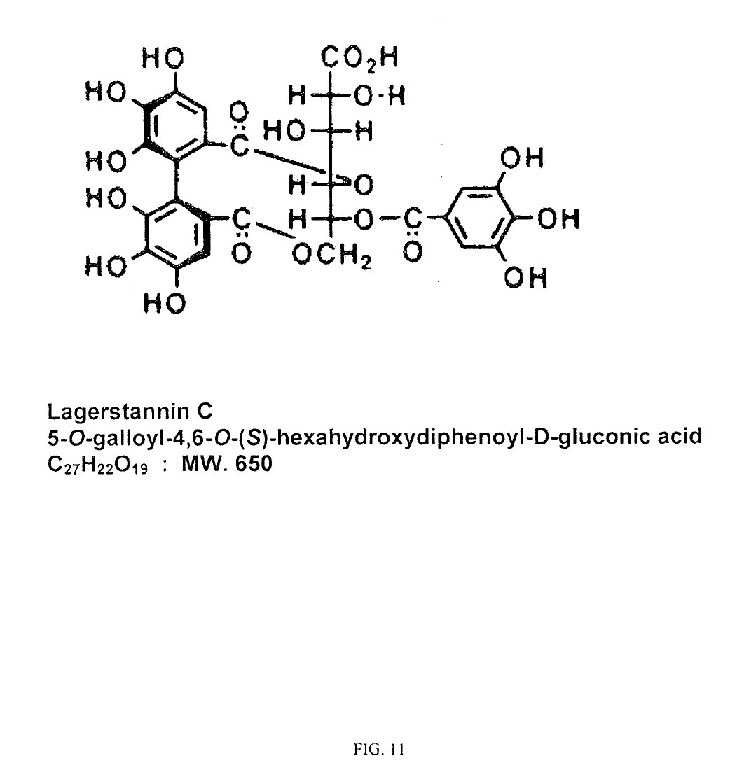

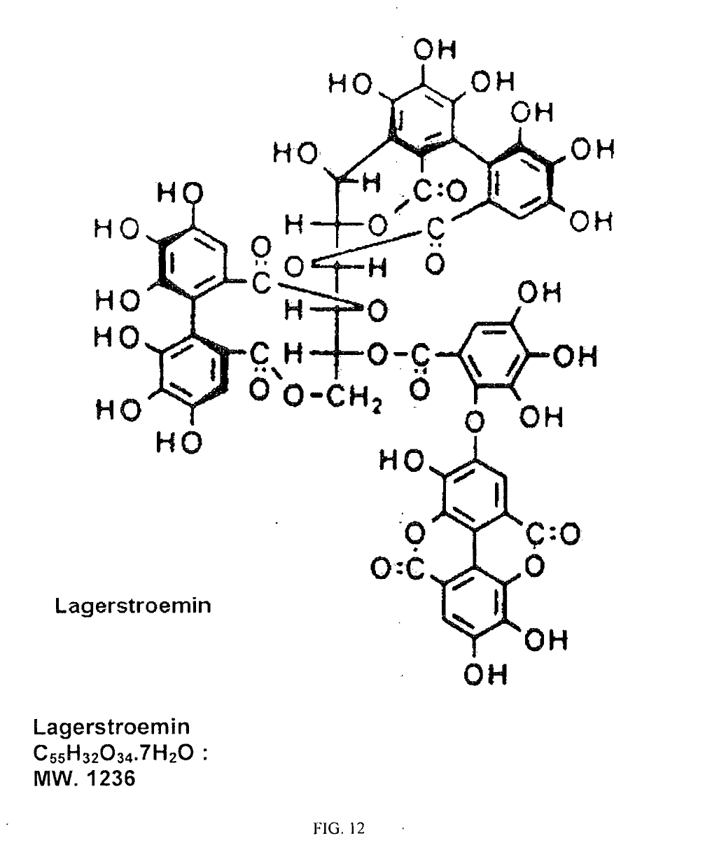

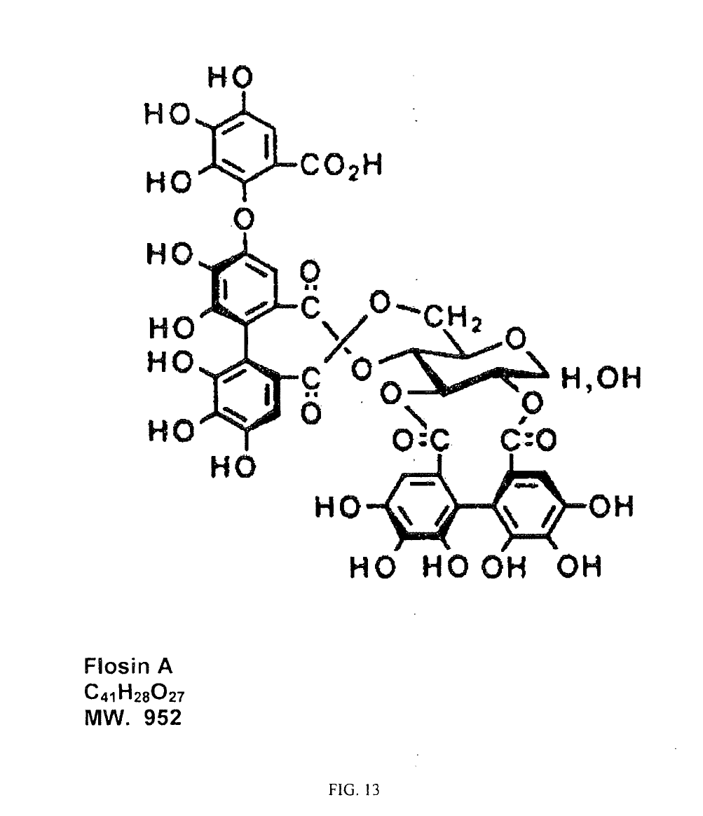

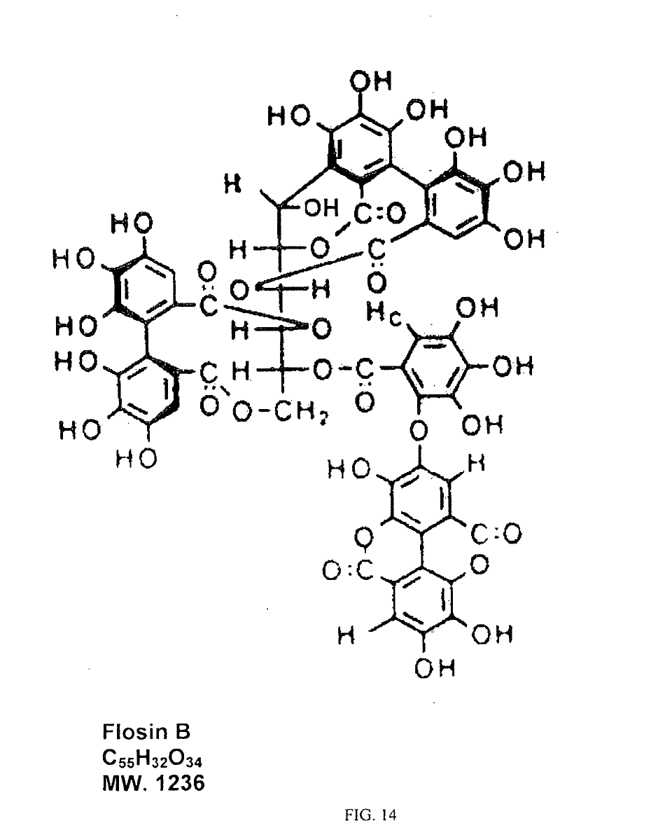

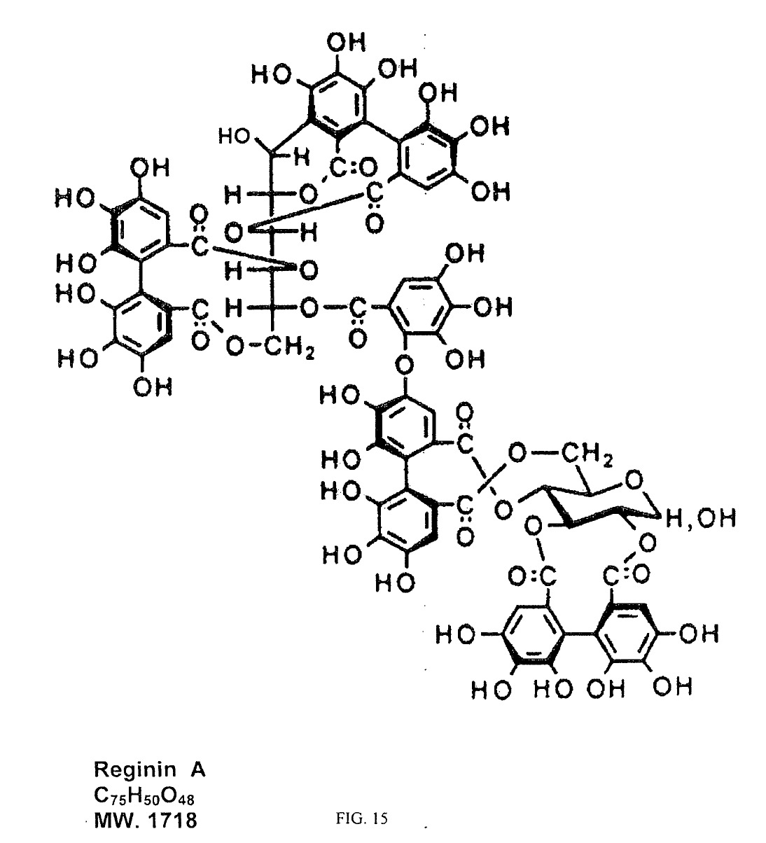

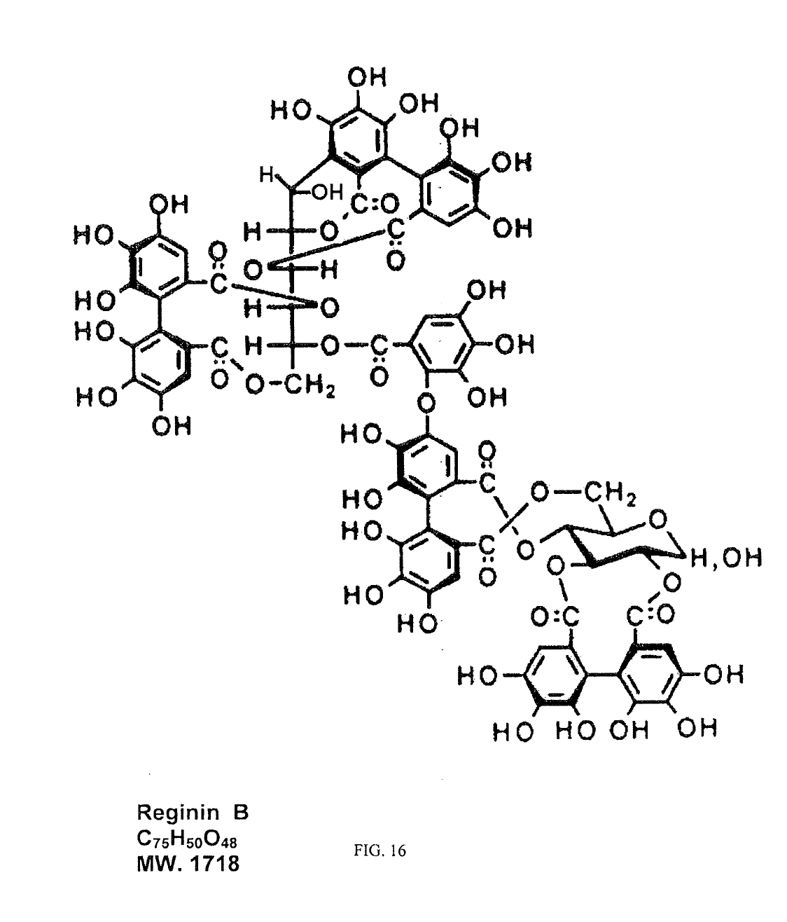

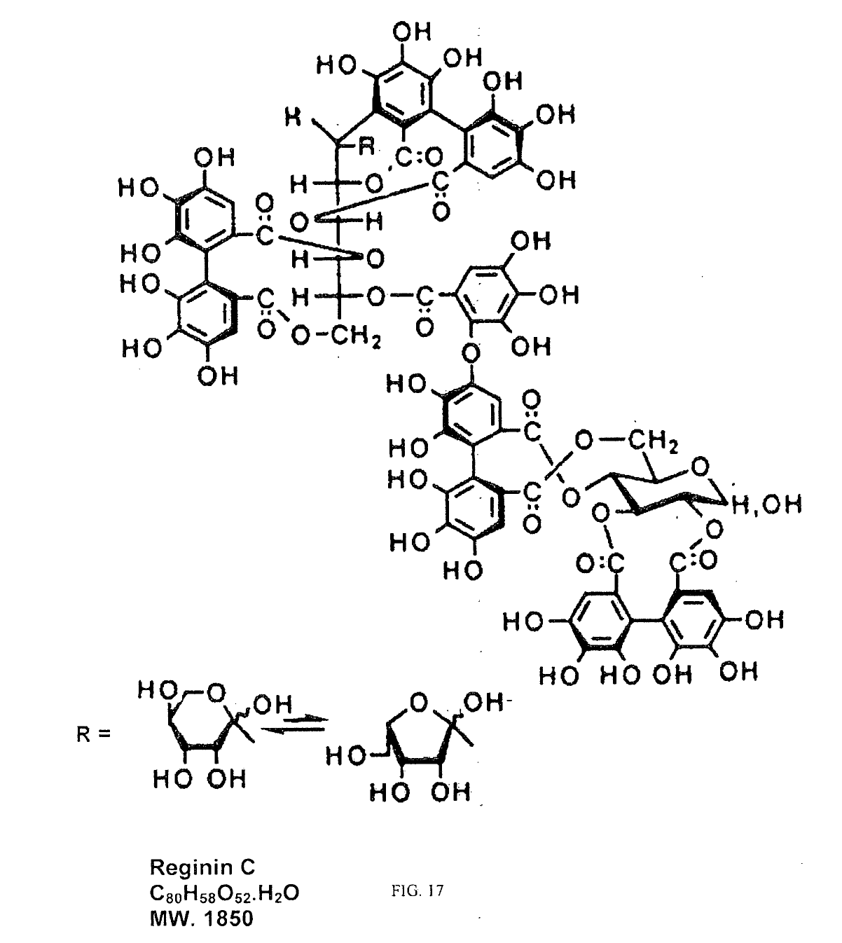

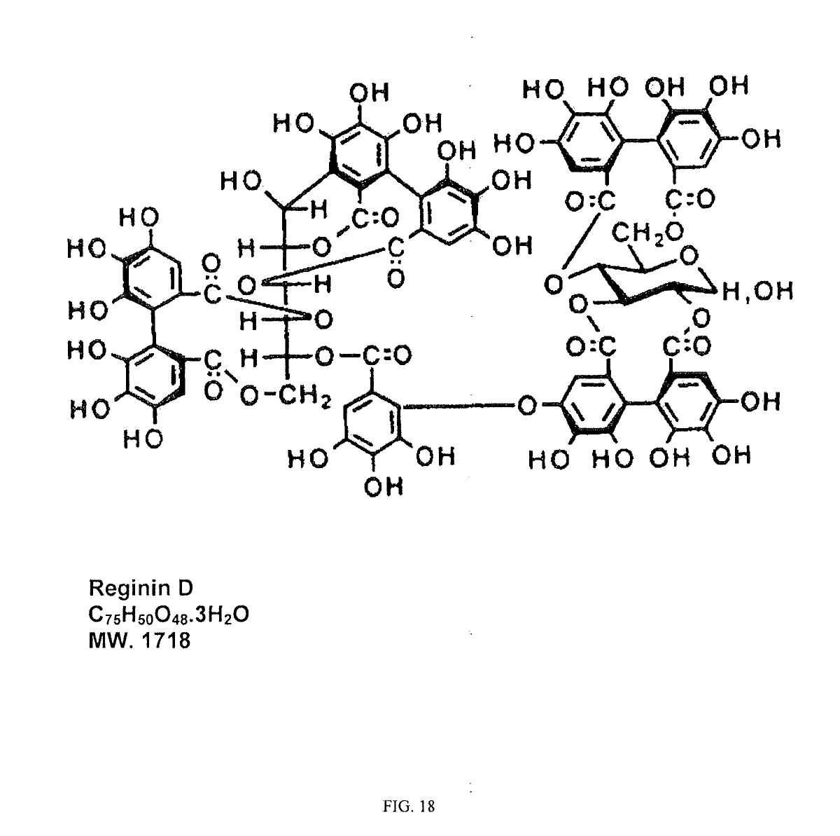

A number of compounds, including various species of tannic acids, have been identified from the Banaba extract by the above-described protocols, that may have potential as active agents, particularly as antihyperglycemic agents. These include Lagerstannin (FIG. 9), Lagerstannin B (FIG. 10), Lagerstannin C (FIG. 11), Lagerstroemin (FIG. 12), Flosin A (FIG. 13), Flosin B (FIG. 14), Reginin A (FIG. 15), Reginin B (FIG. 16), Reginin C (FIG. 17), and Reginin D (FIG. 18).

3. In Vitro Studies of the Banaba Extract

The effect of the banaba plant extracts of the invention can be studied using the D-glucose uptake method disclosed in Murakami C, Myoga K, Kasai R, Ohtani K, Kurokawa T, Ishibashi S, Dayrit F, Padolina W G, Yamasaki K. “Screening of plant constituents for effect on glucose transport activity in Ehrlich ascites tumour cells”, Chem Pharm Bull (Tokyo). December 1993; 41(12): 2129-31. Another approach is that of measuring the transport of labeled D-glucose into cells can be measured by a rapid filtration technique as described by Yamasaki, K. et al. (Phytotherapy Research, 7, 2000, 1993).

4. In Vivo Studies Demonstrating Antihyperglycemic Benefits

Animal Study of Antihyperglycemic Potential of Banaba Extract Formulations

Overview of the anti-Hyperglycemic Study. Six formulations were supplied by inventors to an independent testing facility, in the form of blind-coded formulations: The following overview table identifies formulations tested, dosage, and provides a brief note on the results. Detailed protocol and data follow the Table 1.

TABLE 1
Overview of the study

| | Formulation, | |
|---|---|---|
| | Source, | |
| Code | and Dosage | Anti-Hyperglycemic Potential |
| L 2504019 | Banaba 1% Corosolic | no apparent anti-hyperglycemic |
| | Acid | activity. |
| | Indian raw material | |
| | 9 mg/kg | |
| L 2504020 | Banaba 40% Tannins | moderate anti-hyperglycemic activity |
| | Indian raw material | |
| | 90 mg/kg | |
| L 2504021 | Banaba 20% Tannins | excellent potential agent |
| | Philippine red leaves | |
| | 90 mg/kg | |
| L 2504022 | Banaba 33% Corosolic | no apparent anti-hyperglycemic |
| | Acid | activity |
| | Indian raw material | |
| | 0.27 mg/kg | |
| L 2505024 | Banaba 40% Tannins | excellent potential agent |
| | Philippine red leaves | |
| | 90 mg/kg | |
| L 2506043 | 997 mg 40% Tannins | weak to moderate potential, however |
| | red leaves + 3 mg 33% | this formulation produced a moderate |
| | Corosolic Acid | decrease in body weight in |
| | 90 mg/kg | comparison to control; unlike other |
| | | formulations |

The study was carried out in two parts. In Part I, the test drugs were screened for anti-hyperglycemic effect in rats subjected to sucrose over-loading. Rats in this study were maintained on controlled feeding schedules by providing 15 g pellets per rat. This feeding schedule resulted in mild to moderate decrease in body weight in control rats. In Part II of the study, the test formulations were screened for their effect on body weight and fasting blood sugar level in well-fed conditions.

Part 1 of the study was carried out in Wistar albino rats of either sex. Each group comprised of 8 rats (f4 female and 4 male). The animals were obtained from Sarabhai Research Centre, Vadodara, India, and allowed to acclimatize to the laboratory conditions for 15 days before subjecting them to experimentation. They were maintained on Amrut brand (Pranav Agro Industries, Pune, India) rat pellet feed (control fed condition) and tap water given ad libitum. The animals were maintained at a uniform temperature of 22-24 C. under air-conditioning. They were exposed to 12 h light and 12 h dark cycles. The studies were carried out after obtaining the clearance from the Institutional Animal Ethical Committee as per standard procedures.

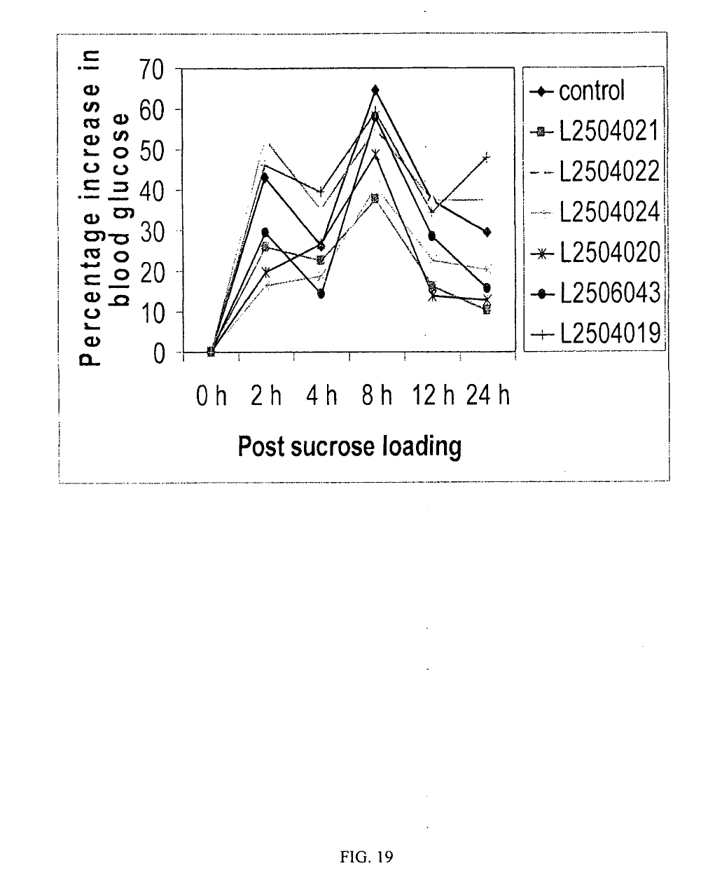

The test drugs were administered for a period of 15 days and after overnight fasting subjected to anti-hyperglycemic study. On the 16th day after recording the initial blood sugar level test drugs were administered to the respective groups. One hour after drug administration, the rats were administered sucrose solution in the dose of 40 g/kg. This was followed by recording of blood sugar level using glucose strip and glucometer (Johnson and Johnson) at various time intervals: 2, 4, 8, 12 and 24 h after sucrose loading; these temporal parameters were standardized on the basis of a pilot study in which it was found that the blood glucose starts rising at 4 hours after sucrose loading and reaching a peak at 8 hr. and then slowly decreasing by 24 h. These results are shown in the time course analysis shown in FIG. 19, and are shown in tabular form, below, in Table 2.

TABLE 2
Anti-hyperglycemic activity in different coded drugs against
sucrose induced hyperglycemia in albino rats

| | Percentage increase in blood sugar at various time intervals | | | | |
|---|---|---|---|---|---|
| Group | 2 h | 4 h | 8 h | 12 h | 24 h |
| Control | 42.89 ± | 25.95 ± | 64.78 ± | 31.59 ± | 29.06 ± |
| | 4.21 | 4.30 | 10.32 | 5.91 | 6.14 |
| L-2504021 | 26.05 ± | 24.42 ± | 37.29 ± | 15.18 ± | 10.35 ± |
| | 7.22 | 8.19 | 09.67 | 3.67* | 6.29 |
| L-2504022 | 52.98 ± | 35.40 ± | 54.44 ± | 37.09 ± | 37.37 ± |
| | 7.37 | 10.85 | 12.44 | 8.43 | 4.94 |
| L-2505024 | 16.88 ± | 16.67 ± | 40.75 ± | 21.21 ± | 13.57 ± |
| | 7.48* | 7.44 | 11.51 | 7.24 | 6.97 |
| L-2504020 | 17.39 ± | 25.77 ± | 47.73 ± | 13.78 ± | 11.94 ± |
| | 5.79** | 3.59 | 05.15 | 5.85 | 3.88* |
| L-2506043 | 29.69 ± | 14.37 ± | 59.78 ± | 23.49 ± | 15.74 ± |
| | 5.38 | 4.35 | 09.77 | 6.15 | 5.27 |
| L-2504019 | 48.04 ± | 33.49 ± | 57.92 ± | 30.57 ± | 28.18 ± |
| | 8.75 | 7.89 | 07.39 | 8.00 | 5.64 |

*P < 0.05;
**P < 0.01 in comparison to control group (Unpaired student's ‘t’ test)

In the control group rats blood sugar level rose by 42.89% at 2 h post sucrose loading, dipped to 25.95% at 4 h and rose again to 64.78% by 8 h and again lowered to around 29% by 24 hours. In L-2504021, the pattern of blood sugar elevation and decline was similar to the one seen in control group except that the magnitude of blood sugar elevation was much less at different time intervals. Further the maximum antagonism of the peak sugar level was observed in this group (42.44% antagonism; see Table 2a).

TABLE 2a

| | Percentage change in blood sugar level (after sucrose | | | | |
|---|---|---|---|---|---|
| | loading) in groups administered the test formulations, | | | | |
| | normalized with respect to the changes observed in | | | | |
| | untreated control group | | | | |
| Group | 2 hr | 4 hr | 8 hr | 12 hr | 24 hr |
| L-2504021 | 39.16 | 5.90 | 42.44 | 51.96 | 64.33 |
| | Decrease | Decrease | Decrease | Decrease | Decrease |
| L-2504022 | 23.65 | 36.43 | 15.95 | 17.41 | 28.53 |
| | Increase | Increase | Decrease | Increase | Increase |
| L-2505024 | 60.64 | 35.76 | 37.03 | 32.85 | 53.48 |
| | Decrease | Decrease | Decrease | Decrease | Decrease |
| L-2504020 | 59.50 | 0 | 26.32 | 56.38 | 58.91 |
| | Decrease | Decrease | Decrease | Decrease | Decrease |
| L-2506043 | 30.48 | 44.62 | 7.71 | 25.64 | 45.84 |
| | Decrease | Decrease | Decrease | Decrease | Decrease |
| L-2504019 | 12.14 | 29.06 | 10.59 | 3.26 | 3.00 |
| | Increase | Increase | Decrease | Decrease | Decrease |

In L-2505022 administered group moderate potentiation of blood sugar elevation after sucrose feeding was observed except for the observation of 15.95% antagonism at the peak period. In L-2505024 administered group antagonism of the sucrose feeding induced blood sugar elevation was observed at all the time intervals at which blood sugar levels were measured. The antagonism was higher in the initial stages in comparison to latter stages. In L-2504020 administered group good antagonism at the initial period, mild to moderate antagonism at the peak period and good antagonism again at the later stages was observed. In L-2506043 administered group moderate to good antagonism in the earlier stages weak antagonism at the peak period and moderate antagonism at the later stages was observed. In L-2504019 administered group moderate potentiation at the earlier part and weak antagonism at the peak period and latter stages was observed.

Part 2 of the study was carried out in Wistar albino rats of either sex. Each group comprised 6 rats, three female and three male. The animals were obtained from Sarabhai Research Centre-Vadodara and allowed to acclimatize to the laboratory conditions for 15 days before subjecting them to experimentation. They were maintained on Amrut brand (Pranav Agro Industries, Pune, India) rat pellet feed and tap water given ad libitum. The animals were maintained at a uniform temperature of 22-24 C. under controlled temperature conditions. They were exposed to 12 h light and 12 h dark light cycles. The studies were carried out after obtaining the clearance from the Institutional Animal Ethical Committee as per standard procedures.

Initially, the blood sugar level was recorded in each rat of all the groups after overnight fasting. The test drugs were administered for a period of 15 days and after overnight fasting blood sugar levels were estimated by glucose strip method using a glucometer as mentioned above. The effect of the test drugs on body weight was recorded at the interval of 5 days. The data obtained have been summarized in Table 3 and Table 4.

TABLE 3
Effect of different coded formulations on the blood sugar level in
normal rats

| | | Blood sugar | |
|---|---|---|---|
| | Initial blood | level at the end | Percentage |
| | sugar level | of the study | decrease in |
| | (mg/dL) | (mg/dL) | blood sugar level |
| Group | mean ± SEM | mean ± SEM | mean ± SEM |
| Control | 56.67 ± 2.55 | 51.66 ± 1.75 | 08.70 ± 2.12 |
| L-2504021 | 64.83 ± 2.71 | 45.50 ± 2.92 | 29.87 ± 3.22** |
| L-2504022 | 66.00 ± 1.69 | 49.83 ± 2.08 | 24.11 ± 4.22* |
| L-2505024 | 58.00 ± 3.61 | 53.16 ± 3.75 | 10.55 ± 5.35 |
| L-2504020 | 60.33 ± 3.04 | 56.16 ± 2.05 | 06.39 ± 2.50 |
| L-2506043 | 68.15 ± 4.50 | 53.00 ± 4.94 | 22.03 ± 3.14** |
| L-2504019 | 62.83 ± 3.78 | 54.65 ± 2.65 | 13.46 ± 5.98 |

*P < 0.05;
**P < 0.01 in comparison to control group (Unpaired student's ‘t’ test)

In control group, 8.70% decrease in blood sugar level was observed in comparison to initial values. In L-2504021, 29.87% decrease was observed which was found to be statistically significant in comparison to the control group. Similar effect was observed with L-2504022 and L-2506043, although the decrease was slightly less (24.11% and 22.03%, respectively). The mild to moderate decrease observed with L-2505024, L-2504020 and L-2504019 was found to be statistically non-significant in comparison to control group.

TABLE 4
Effect of different coded formulations on the body weight in albino rats

| | Percentage increase in body weight at various | | |
|---|---|---|---|
| | time intervals (mean ± SEM) | | |
| Group | 5^{th} day | 10^{th} day | 15^{th} day |
| Control | 15.72 ± 1.97 | 21.24 ± 0.83 | 27.33 ± 1.74 |
| L-2504021 | 11.05 ± 1.90 | 15.74 ± 1.60* | 22.47 ± 1.68 |
| L-2504022 | 14.75 ± 2.22 | 15.73 ± 2.17* | 18.48 ± 1.93** |
| L-2505024 | 20.71 ± 0.78 | 20.71 ± 0.78 | 24.93 ± 1.80 |
| L-2504020 | 16.65 ± 1.74 | 18.24 ± 1.59 | 26.53 ± 2.13 |
| L-2506043 | 16.35 ± 1.73 | 24.04 ± 0.85 | 27.91 ± 1.77 |
| L-2504019 | 19.52 ± 1.44 | 22.13 ± 1.64 | 23.14 ± 1.55 |

*P < 0.05;
**P < 0.01 in comparison to control group (Unpaired student's ‘t’ test)

In control group rats, consistent and gradual body weight gain was observed over a period of 15 days observation period. The body weight gain was observed in all the drug formulations administered groups in comparison to initial values. In L-2504021 and L-2504022 formulation administered groups, the body gain observed was less in comparison to control group in magnitude, indicating interference with body weight gain. The body weight gain was significantly less with respect to the data recorded at 10 days in case of L-2504021 and with respect to data recorded at both the 10th and 15th days in case of L-2504022. In the remaining groups the body weight change did not show any significant difference in comparison to control group.

Comments on the animal study data: The formulations were prepared with an idea of obtaining a test formulation that would have efficacy in the treatment of obese diabetic persons. The drug would ideally have very good anti-hyperglycemic activity against glucose or sucrose loading, minimal or weak anti-hyperglycemic activity in normal animals, and good body weight-decreasing activity. At present, the main focus for treatment of NIDD diabetes is to have a drug, which prevents the sudden increase in postprandial blood sugar level and such drugs are still better if they have minimum effect on normal blood sugar level implying that overdose would not produce drug induced hypoglycemia. If such a drug has weight reducing effect it would be an added advantage in treating obese diabetic patients.

Against this background the 6 test formulations were screened for anti-hyperglycemic, hypoglycemic and body weight gain-decreasing or -retarding effect. For the treatment of diabetes the most suitable drug is one that produces good and long lasting anti-hyperglycemic effect. If it possess weight decreasing effect it would be an added advantage. If it does not possess hypoglycemic in addition to the above effects it would be a most ideal one. Thus any selection of ideal formulation would have to start from the presence of anti-hyperglycemic activity, this being first criterion. In this activity, the drugs, which antagonize the peak raise in blood sugar level and sustain it for longer period, are normally preferred.

Based on this criteria, of the 6 formulations (statistical significance is ignored since many effects observed are nearly significant and the emphasis is on the magnitude of anti-hyperglycemic effect; Table 1a.), L-2504021 and L-2505024 emerge as the best anti-hyperglycemic agents. L-2504020 ranks next with moderate anti-hyperglycemic effect at peak level but good antagonism in the latter part. L-2506043 with weak to moderate activity is ranked next. The main draw back is that it has weak antagonism at the peak period. L-2504022, because it produces higher blood sugar level after sucrose loading in comparison to normal control rats, indicating pro-hyperglycemic activity at the dose level studied, should have no place in an anti-diabetic formulation. L-2504019, because of its tendency to produce pro-glycemic effect in the initial part and very weak antagonism later, also is not suitable for any formulations intended for administration to diabetic patients.

The second criterion is the presence of body weight reduction. None of the test formulations produced weight reduction (under well-fed conditions) if initial body weight is taken as the base point. If body weight gain pattern is taken into consideration, then the obtained results indicate that L-2504022 has good body weight-gain retarding activity; hence, it would be a candidate for weight reduction but not in obese diabetes patients. The other formulation with moderate body weight gain-retarding effect is L-2504021. The fact that it also has very good anti-hyperglycemic activity makes it the best of the formulations tested for the treatment of obese diabetes patients.

The third criterion is that the drug which has the above attributes (anti-hyperglycemic and weight gain-retarding effects) should not produce strong hypoglycemic effect in normoglycemic rats. L-2504021 does not fully meet this criterion because it produces moderate [strong] hypoglycemic effect [in normoglycemic rats]. L-2504022, surprisingly, produced a pro-glycemic effect in sucrose-loaded rats and produced moderate hypoglycemic activity in normoglycemic rats. L-2506043 also produced moderate hypoglycemic effect. Since it has no weight gain-retarding effect and has only a modest anti-hyperglycemic effect, it does not warrant immediate attention for further development. L-2505024 and L-2504020 did not produce significant hypoglycemic activity. Since they possess moderate anti-hyperglycemic activity they may be candidates for developing as agents for the treatment of non-obese diabetes, but they may not be suitable for the treatment with the aim of reducing body weight.

Taking all the factors into consideration, L-2504021 can be considered as the best candidate formulation for further study and product optimization. L-2505024 also merits another consideration for further study.

General Testing Protocol and Types of Human Subjects

Anti-diabetic activity of the standardized extracts of the invention can be measured following the protocol set forth by Judy, William V., et al. (“Antidiabetic activity of a standardized extract (Glucosol™) from Lagerstroemia speciosa leaf in Type II diabetics—A dose-dependence study. Journal of Ethnopharmacology” 87 (2003) 115-117). Basically, the leaves of banaba plants can be harvested and extracted into groups. However, instead of being dissolved in an alcohol, the end extract products can be purified by other methods well known in the art, such as treatment with activated carbon to remove impurities (such as chlorophyll), filtered, and concentrated under reduced pressure to give dry solids. The concentration of the active agents (corosolic acid, gallotannins, or ellagitannins) can be determined by HPLC, as described in the protocol above. The extracts from each sample and each group can then be standardized to desired specifications. The standardized end products can then be formulated into tablets, soft- or hard gel tablets, or into other suitable carriers as described in the specification above.

The standardized and formulated compositions of the invention can then be administered to volunteers suffering from disorders of glucose metabolism, such diabetes, hyperglycemia, Syndrome X, obesity, or the like in the dosages determined in accordance with the invention. Volunteers will be separated into 6 sets of two groups of equal numbers (one group in each set will be given the active agent from groups 1, 2, 4, 8, 9, and 10 and the other group will be given a placebo). Basal blood glucose levels will be determined before administration of the formulations or placebos. Each group will receive an oral dosage for 15 days at differing quantities, with a reasonable wash out period between the doses. Blood samples will be taken regularly 7 days before the first administration and regularly through out the 15 day period after administration begins. Blood glucose levels will be determined by means well known in the art, such as by use of a clinical glucose monitor, and will be graphed against blood glucose levels determined in the 7 day period before administration began. Statistical analysis will then determine the effect of each component of groups 1, 2, 4, 8, 9, and 10 vs. the placebos on blood glucose levels over the dose range.

Equivalents of the Invention

While a number of particular embodiments of the invention and variations thereof have been described in detail, other modifications and methods of using the disclosed therapeutic combinations will be apparent to those of skill in the art. Accordingly, it should be understood that various applications, modifications, and substitutions may be made of equivalents without departing from the spirit of the invention or the scope of the claims. Various terms and conventions have been used in the description to convey an understanding of the invention. It will be understood that a corresponding description of these various terms applies to common linguistic or grammatical variations or forms of these various terms. It will also be understood that some products have been identified by trade names, but that these names are provided as contemporary examples, and the invention is not limited by such literal scope, particularly when such products or compounds have been described in chemical terms. Although the written description offers biochemical theory and interpretation of available data in describing the invention, it should be understood that such theory and interpretation do not bind or limit the claims. Further, it should be understood that the invention is not limited to the embodiments set forth herein for purposes of exemplification, but is to be defined only by a fair reading of the appended claims, including the full range of equivalency to which each element thereof is entitled.

1. A composition derived from Lagerstroemia speciosa comprising corosolic acid and one or more compounds selected from the group consisting of gallotannins, ellagitannins and valoneic acid dilactone.

2. The composition of claim 1, wherein the concentration of corosolic acid is between about 15.5% to about 98.5%.

3. The composition of claim 1, wherein the concentration of corosolic acid is between about 20% to about 80%.

4. The composition of claim 1, wherein the combined concentration of gallotannins and ellagitannins is between about 2.5% and about 98%.

5. The composition of claim 1, wherein the combined concentration of gallotannins and ellagitannins is between about 5% and about 90%.

6. The composition of claim 1, wherein the concentration of valoneic acid dilactone is between about 0.1% and about 20%.

7. The composition of claim 1, wherein the concentration of valoneic acid dilactone is between about 1% and about 10%.

8. A composition derived from Lagerstroemia speciosa comprising one or more compounds selected from the group consisting of gallotannins, ellagitannins and valoneic acid dilactone.

9. The composition of claim 8 wherein the combined concentration of gallotannins and ellagitannins is between about 2.5% and about 98%.

10. The composition of claim 8 wherein the combined concentration of gallotannins and ellagitannins is between about 5% and about 90%.

11. The composition of claim 8 wherein the combined concentration of gallotannins and ellagitannins is between about 10% and about 60%.

12. The composition of claim 8 wherein the combined concentration of gallotannins and ellagitannins is between about 35% and about 45%.

13. A composition derived from Lagerstroemia speciosa consisting essentially of one or more compounds selected from the group consisting of gallotannins, ellagitannins and valoneic acid dilactone, said composition being essentially free of corosolic acid.

14. The compositions of claim 1 or claim 8, wherein the composition is 99% water soluble.

15. The compositions of claim 1 or claim 84 wherein the composition is a therapeutic agent sufficient for the treatment of a disorder of glucose metabolism.

16. The compositions of claim 1 or claim 8, wherein the composition is a therapeutic agent sufficient for the treatment of hyperuricemia.

17. The compositions of claim 1 or claim 8, wherein the composition is combined with a second therapeutic agent sufficient for the treatment of disorder of glucose metabolism.

18. The compositions of claim 1 or claim 8, wherein the composition is a therapeutic agent sufficient for the treatment of hyperuricemia.

19. A method of treating hyperglycemia comprising administering to a hyperglycemic subject an effective dose of composition derived from Lagerstroemia speciosa, the composition comprising corosolic acid and one or more compounds selected from the group consisting of gallotannins, ellagitannins and valoneic acid dilactone.

20. A method of treating hyperglycemia comprising administering to a hyperglycemic subject an effective dose of composition derived from Lagerstroemia speciosa, the composition comprising one or more compounds selected from the group consisting of gallotannins, ellagitannins and valoneic acid dilactone.

21. A method of extracting a composition derived from Lagerstroemia speciosa, the composition comprising corosolic acid and one or more other compounds selected from the group consisting of gallotannins, ellagitannins and valoneic acid dilactone, the method comprising controlling the final concentration of each compound independently of the other compounds, such control being provided by application of solvents of varying polarities to create separate fractions, and by blending of the separately extracted fractions.

22. A method of extracting a composition derived from Lagerstroemia speciosa, the composition comprising one or more compounds selected from the group consisting of gallotannins, ellagitannins and valoneic acid dilactone, the method comprising controlling the final concentration of each compound independently of the other compounds, such control being provided by application of solvents of varying polarities to create separate fractions, and by blending of the separately extracted fractions.
